(19) United States
(12) Patent Application Publication
(10) Pub. No.: US 2018/0094248 A1
(43) Pub. Date: Apr. 5, 2018

(54) Necroptosis Signaling as a Therapeutic Target for Alzheimer's Disease

(71) Applicant: Arizona Board of Regents on behalf of Arizona State University, Scottsdale, AZ (US)

(72) Inventor: Salvatore Oddo, Tempe, AZ (US)

(21) Appl. No.: 15/638,895

(22) Filed: Jun. 30, 2017

Related U.S. Application Data
(60) Provisional application No. 62/356,983, filed on Jun. 30, 2016.

(51) Int. Cl. C12N 9/12; C07K 14/47; A61K 38/43; C07K 16/28; C07K 16/44; A61K 48/00; A61K 38/00; C12Q 1/00

(52) U.S. Cl. CPC C12N 9/12; C07K 14/47; A61K 38/43; C12Q 1/00; C07K 16/44; A61K 48/00; A61K 38/00; C07K 16/28

(57) ABSTRACT

Therapeutic methods for conditions associated with neuronal loss and necroptosis are provided. More particularly, provided herein are methods of treating neuronal loss in a subject having or suspected of having a condition associated with neuronal loss.

### CROSS-REFERENCE TO RELATED APPLICATIONS

This application claims the benefit of U.S. Provisional Application No. 62/356,983, filed Jun. 30, 2016, which is hereby incorporated by reference in its entirety for all purposes.

### STATEMENT REGARDING FEDERALLY SPONSORED RESEARCH

Not applicable.

### BACKGROUND

Alzheimer's disease (AD), the most common neurodegenerative disorder, is characterized by severe neuronal loss. Imaging studies and postmortem examinations of Alzheimer's disease (AD) brains have consistently showed a striking reducing in brain volume and cell number, respectively. Despite the tremendous progress made towards the understanding of the pathogenesis of AD, the mechanisms underlying neuronal loss remain elusive.

Accordingly, there remains a need for a better understanding of the etiopathology of neurodegenerative diseases. In addition, there remains a need in the art for improved methods for detecting neuronal loss and for treating conditions associated with neuronal loss by administering compounds that slow, halt, or reverse necroptosis.

### SUMMARY

In a first aspect, provided herein is method of reducing neuronal loss in a subject having or suspected of having a neurodegenerative disease, the method comprising the step of administering to the subject a therapeutically effective amount of a compound that inhibits a necroptosis-associated activity of receptor-interactive protein kinase 1 (RIPK1), RIPK3, or Mixed Lineage Kinase Domain-like (MLKL), or a pharmaceutically acceptable salt thereof, whereby neuronal loss is reduced in the subject. The compound can be a small molecule inhibitor of RIPK1, RIPK3, or MLKL. The small molecule inhibitor can be 7-Cl-O-necrostatin-1S (Nec-1S), necrosulfonamide, or GSK'872, or a pharmaceutically acceptable salt thereof. The compound can inhibit phosphorylation of MLKL at an amino acid position selected from the group consisting of position 357 and position 358, wherein the position is numbered relative to SEQ ID NO:2. The compound can inhibit formation of MLKL homodimers. The neurodegenerative disease can be a neurodegenerative disease of the central or peripheral nervous system. The neurodegenerative disease can be selected from the group consisting of Alzheimer's disease (AD), multiple sclerosis (MS), Huntington Disease (HD), amyotrophic lateral sclerosis (ALS), and Down Syndrome.

In another aspect, provided herein is a method of treating neuron loss in a subject having or suspected or having a condition associated with aberrant necroptosis activation, the method comprising administering to the subject a therapeutically effective amount of a compound that inhibits a necroptosis-associated activity of RIPK1, RIPK3, or MLKL, or a pharmaceutically acceptable salt thereof, whereby neuronal loss is reduced in the subject. The compound can be a small molecule inhibitor of RIPK1, RIPK3, or MLKL. The small molecule inhibitor can be 7-Cl-O-necrostatin-1S (Nec-1S), necrosulfonamide, or GSK'872, or a pharmaceutically acceptable salt thereof. The compound can inhibit phosphorylation of MLKL at an amino acid position selected from the group consisting of position 357 and position 358, wherein the position is numbered relative to SEQ ID NO:2. The compound can inhibit formation of MLKL homodimers. The condition associated with aberrant necroptosis activation can be a neurodegenerative disease of the central nervous system or peripheral nervous system. The neurodegenerative disease can be selected from the group consisting of Alzheimer's disease (AD), multiple sclerosis (MS), Huntington's Disease (HD), Parkinson's Disease (PD), amyotrophic lateral sclerosis (ALS), and Down Syndrome.

In a further aspect, provided herein is use of a therapeutically effective amount of a compound that inhibits receptor-interactive protein kinase 1 (RIPK1) or Mixed Lineage Kinase Domain-like (MLKL), or a pharmaceutically acceptable salt thereof, in a dosage sufficient to decrease neuronal loss in a subject having a neurodegenerative disease. The neurodegenerative disease can be Alzheimer's disease (AD), multiple sclerosis (MS), Huntington's Disease (HD), Parkinson's Disease (PD), amyotrophic lateral sclerosis (ALS), or Down Syndrome.

The foregoing and other advantages of the invention will appear from the following description. In the description, reference is made to the accompanying drawings, which form a part hereof, and in which there is shown by way of illustration a preferred embodiment of the invention. Such embodiment does not necessarily represent the full scope of the invention, however, and reference is made therefore to the claims and herein for interpreting the scope of the invention.

### BRIEF DESCRIPTION OF DRAWINGS

The invention will be better understood and features, aspects, and advantages other than those set forth above will become apparent when consideration is given to the following detailed description thereof. Such detailed description makes reference to the following drawings.

The patent or application file contains at least one drawing executed in color. Copies of this patent or patent application publication with color drawing(s) will be provided by the Office upon request and payment of the necessary fee.

FIGS. 1a-1g demonstrate an increase in necroptosis markers in AD human brains. a, Representative western blots of proteins extracted in RIPA and urea buffers from AD (n=12 cases) and CTL (n=11 cases) brains probed with the indicated antibodies. b-c, Quantitative analyses of the blots showed elevated levels of RIPK1 [t(21)=3.444, p=0.002 for RIPA; t(21)=2.205, p=0.038 for urea] and MLKL [t(21)=2.443, p=0.023 for RIPA; t(21)=3.126; p=0.0047 for urea] in AD patients compared to CTL in both fractions. d, Representative microphotographs of brain sections from AD (n=14 cases) and CTL (n=12 cases) patients immunostained with the indicated antibodies. e-g, Quantitative analyses of the immunoreactivity showed a significant increase of RIPK1 and MLKL in AD patients compared to CTL [t(24)=3.447, p=0.002 and t(24)=2.667, p=0.013, respectively]. No significant difference was detected in RIPK3 levels [t(24 0.0001, p=0.999]. Quantification of western blots were obtained by normalizing the protein of interest to β-actin. Data are presented as box plots, and were analyzed by unpaired t-test.

FIGS. 2a-2m demonstrate necrosome formation in Alzheimer's disease. a-f, Microphotographs and quantitative analyses of brain sections from AD and CTL patients immunostained with the indicated antibodies. Quantitative analysis showed that the number of co-localized pixels was significantly higher in AD patients compared to control [panel b: t(28)=3.270, p=0.003; panel d: t(28)=2.659. p=0.013; panel f: t(28)=2.437, p=0.022). g, Proteins extracted from AD and CTL brains were immunoprecipitated with an antibody against RIPK1 and probed for MLKL. The IgG band is shown as a control. The graph shows the quantification of the western blots and highlights a stronger interaction between RIPK1 and MLKL in AD brains compared to CTL [t(12)=2.542, p=0.026]. h, Microphotographs of brain sections immunostained with a pMLKL specific antibody. i, Quantitative analysis of the pMLKL immunohistochemistry showed a significant increase of pMLKL in AD patients compared to CTL [t(22)=3.277 (p=0.003]. j, Representative western blot run in non-reducing conditions probed with a MLKL antibody. The two bands represent MLKL monomers and dimers. k, Quantitative analyses of the MLKL blots was obtained by normalizing the intensity value of the dimers over the intensity value of the monomers [t(22)=2.322, p=0.030]. (1) Microphotographs of brain sections from AD and CTL patients co-labeled with the indicated antibodies. Cadherin is used as a membrane marker. Statistical evaluation by Mander's correlation, followed by Costes randomization test indicates that in CTL cases, 36.47±1.4% of pMLKL immunoreactivity was located in the membrane (R=0.3288 and Costes p=0.96). In AD cases, 42.31±1.5% of pMLKL immunoreactivity was located in the membrane (R=0.360 and Costes p=0.98). (m), Quantitative analyses of the pMLKL/cadherin co-localization showed that the number of co-localized pixels was significantly higher in AD cases compared to CTL cases [t(28)=4.504, p=0.0001]. Data in panels b, d, f, g, i, k, and m are presented as box plots, and were analyzed by unpaired t-test. n=15 cases for CTL and n=15 cases for AD for panels a-f, and l-m; n=11 cases for CTL and n=13 cases for AD for panels h-k; n=7 cases for CTL and n=7 cases for AD for panel g.

FIGS. 3a-3d demonstrate that activation of necroptosis in a mouse model of AD is linked to reduced brain weight. a-c, Boxplot of Log2 expression values in AD patients (AD) and controls (CTL) for genes RIPK1, RIPK3, and MLKL, respectively. The data indicate that RIPK1 and MLKL levels were significantly higher in AD compared to CTL cases [t(193)=6.890, p=7.5E-11; and t(193)=7.017, p=3.7E-11, respectively]. In contrast, RIPK3 levels were not different between the two groups [t(193)=−0.247, p=0.805]. d. Scatterplot and regression line for the regression (without covariates) between RIPK1 and brain weight with 95% confidence intervals (shaded area). Data in panels a-c are presented as box plots, and were analyzed by moderated t-test (n=98 cases for CTL and n=97 cases for AD). Data in panel d were analyzed by linear regression (n=93 cases for AD).

FIGS. 4a-4i demonstrate that RIPK1 and MLKL activation correlate with Braak stage. a-c, Boxplot of Log2 expression values in AD patients for each Braak stage for RIPK1, RIPK3, and MLKL, respectively. Outliers were computed with the interquartile range rule (IRQ). RIPK1 and MLKL expression levels positively correlate with Braak stage [t(92)=2.107, p=0.035; t(92)=2.488 and p=0.013, respectively]. RIPK3 levels did not correlate with Braak stage [t(92)=0.677, p=0.499]. Data were analyzed by ordinal logistical regression and presented as box plots. d-e, Microphotographs of AD brain sections co-labeled with the indicated antibodies. Statistical evaluation by Mander's correlation followed by Costes randomization test indicated that 55.48±2.8% and 46.85±4.0% of the AT8 immunoreactivity co-localized with RIPK1 (R=0.605, Costes p=0.99) and pMLKL (R=0.529, Costes p=0.98), respectively. f-i, Results for the quantile regression between MMSE and the expression levels of the three necroptotic markers, RIPK1, RIPK 3, and MLKL. The scatter plots show the regression coefficients as a function of percentiles and the standard errors (shaded area). For RIPK1, after FDR correction there was a negative significant regression for percentiles ranging from 30th to the 40th. For RIPK3, no significant correlation was detected for any of the percentiles. For MLKL, after FDR correction there was a negative significant regression for all percentiles ranging from the 30th to the 50th. g, Results for the quantile regression between MMSE and RIPK1:MLKL. After FDR correction, the interaction RIPK1:MLKL negatively correlates with MMSE for a wider range of percentiles (20th-50th). n=94 AD cases for panels a-c, n=15 for panels d-e, and n=62 for panels f-i.

FIG. 5 is a schematic representation of regulation of AD transcriptome and risk associated genes by RIPK1. Genes regulated by RIPK1 overlap significantly with multiple AD expression profiles and brain disease risk loci. Interactions detected in either anterior prefrontal cortex or ectorhinal cortex shown. Red edges indicate genes positively regulated by RIPK1, blue denotes negative regulation. (AD: Alzheimer's disease, ALS: Amyotrophic Lateral Sclerosis, ID: Intellectual disability, ASD: Autism Spectrum Disorder, SCZ: Schizophrenia, ADHD: Attention Deficit Hyperactivity Disorder).

FIGS. 6a-6k demonstrate that necroptosis activation exacerbates cognitive deficits in APP/PS1 mice. a-b, Learning curves of mice trained in the spatial reference version of the Morris water maze (MWM). NonTg-GFP, n=14 mice; NonTg-MLKL, n=12 mice; APP/PS1-GFP, n=14 mice; APP/PS1-MLKL, n=14 mice. The escape latency and the distance traveled to find the hidden platform was plotted against the days of training. The values for each day represent the average of four training trails. For the escape latency, day effect [F(4, 250)=31.26; p<0.0001], group effect [F(3, 250)=21.37; p<0.001], and group x day interaction [F(12, 250)=2.16; p=0.014]. For distance traveled, day effect [F(4, 250)=45.48; p<0.0001], group effect [F(34, 250)=31.46; p<0.001], and group x day interaction [F(12, 250)=1.87; p=0.040]. Post hoc tests indicated that the escape latency for APP/PS1-MLKL mice was significantly different than NonTg-GFP mice at days 3, 4, and 5 (depicted with the # symbol); it was significantly different than NonTg-MLKL and APP/PS1-GFP mice at day 5 (depicted with the ## and ### symbols, respectively). Post hoc tests indicated that the distance traveled for APP/PS1-MLKL mice was significantly different than NonTg-GFP mice at days 2-5 (depicted with the # symbol); it was significantly different than NonTg-MLKL mice at days 3 and 4 (depicted with the ## symbol); it was significantly different than APP/PS1-GFP mice at days 2, 4, and 5 (depicted with the ### symbol). c, Number of platform location crosses during a single 60-second probe trial [F(3, 50)=11.59; p<0.0001]. Post hoc tests showed that NonTg-GFP performed significantly better than the other three groups (depicted with the ‡ symbol). Also, APP/PS1-MLKL performed significantly worse than the other three groups (depicted with the ₄ symbol). d, Number of platform location crosses analyzed as a percentage change indicated that the slopes for the two groups are significantly different from each other [F(1, 38)=4.67; p=0.037]. e, Swim speed measured during a single 60-second probe trial. The values were not statistically significant among the groups [F(3, 50)=1.164; p=0.333]. f-g, Microphotographs of NonTg brain sections injected with the GFP or the MKLK virus, respectively. Sections were stained with an anti-GFP antibody to visualize viral diffusion. h-j, Sections from APP/PS1-GFP mice (n=8) were stained with the indicated antibodies. Mander's correlation analysis indicated a 66.84±3.2% co-localization between GFP and the neuronal maker NeuN, 20.06±3.0% co-localization between GFP and the astrocytic marker GFAP, and 31.14±2.4% co-localized between GFP and the microglial marker Iba1. k, Quantitative analysis of the co-localized pixels indicated that most of the virus injected neurons. One way ANOVA indicated that these values were significantly different among each other [F(2, 23)=70.35; p<0.001]. Bonferroni's multiple comparison test indicated that all three groups were statistically different from each other. Data in panels a-b were analyzed by two-way ANOVA and are presented by mean±s.e.m; Data in panels c, e, and k were analyzed by one-way ANOVA and are presented as box blots. Bonferroni's was used for post hoc tests. Data in panel d were analyzed by linear regression and are presented as mean±s.e.m.

FIGS. 7a-7c demonstrate that constitutively active MLKL induces a higher degree of neuronal death in APP/PS1 mice than NonTg mice. a, Representative CA1 sections from APP/PS1 and NonTg mice injected with the MLKL or GFP AAVs and stained with NeuN, a neuronal marker. b, Quantitative analyses of the stained sections (n =18 pictures per group: 3 pictures per mouse, 6 mice per group). Data were analyzed by one-way ANOVA and presented as box plots. Analyses revealed a significant difference among the four groups [F(3, 67)=21.77; p<0.0001]. Bonferroni's post hoc analyses indicated that all the pairwise comparisons were significantly different with the exception of NonTg-GFP and APP/PS1-GFP. c, Analysis of the stained sections presented as percentage change. Data were analyzed by linear regression, which indicated that the slopes for the two groups are significantly different from each other [F(1, 67)=4.79; p=0.032].

FIGS. 8a-8g demonstrate that necroptosis contributes to neuronal death in 5xFAD mice. a, Primary neurons from APP/PS1 and wild type mice were treated with Nec-1S or vehicle (APP/PS1: n=36 wells from 9 mice per drug/timepoint; WT: n=12 wells from 3 mice per drug/timepoint). The graph shows the number of NeuN-positive neurons after 11 and 15 DIV. Using a two-way ANOVA, we found that there was a significant effect for time [F(1,184)=76.13; p<0.0001] and groups [F(3,184)=7.109; p=0.0002] as well as a significant time x group interaction [F(3,184)=7.109; p=0.0002]. b, The levels of total and phosphorylated MLKL at DIV 15 were measured by western blots (APP/PS1-veh, n=5; APP/PS1-Nec1s, n=5; WT-veh, n=4; WT-Nec1s, n=4). The graph shows the quantitative analyses of the blots as a ratio pMLKL/MLKL. Data were analyzed by one-way ANOVA and presented as box plots. Analyses revealed a significant difference among the four groups [F(3,14)=10.20; p=0.0008]. Bonferroni's post hoc analyses indicated that all the APP/PS1-veh group was significantly different from the other three groups. c, Primary neurons from APP/PS1 and wild type mice were infected with an AAV expressing GFP and then treated with Nec-1S or vehicle (APP/PS1: n=72 wells from 9 mice per drug/timepoint; WT: n=16 wells from 2 mice per drug/timepoint). The graph shows the GFP fluorescence after 11 and 15 DIV. Using a two-way ANOVA, we found that there was a significant effect for time [F(1,344)=170.2; p<0.0001] and groups [F(3,344)=102.4; p<0.0001) as well as a significant time x group interaction (F(3,344)=78.43; p<0.0001). d, The levels of total and phosphorylated MLKL at DIV 15 were measured by western blots (APP/PS1-veh, n=5; APP/PS1-Nec-1S, n=5; WT-veh, n =4; WT-Nec-1S, n=4). The graph shows the quantitative analyses of the blots as a ratio pMLKL/MLKL. Data were analyzed by one-way ANOVA and presented as box plots. Analyses revealed a significant difference among the four groups [F(3,14)=11.30; p=0.0005]. Bonferroni's post hoc analyses indicated that all the groups were significantly different from the APP/PS1-veh. e, Representative brain sections from 5xFAD mice treated with Nec-1S or vehicle stained with Fluoro-Jade. f, Quantitative analyses of the Fluoro-Jade staining. Data were analyzed by unpaired t-test and presented as box plots. Analyses revealed a significant difference between groups [t(7)=4.353; p=0.0033]. g, The levels of total and phosphorylated MLKL in Nec-1S and vehicle-treated 5xFAD mice were measured by western blots. The graph shows the quantitative analyses of the blots as a ratio pMLKL/MLKL. Data were analyzed by unpaired t-test and presented as box plots. Analyses revealed a significant difference between groups [t(7)=2.627; p=0.0341].

FIGS. 9a-9d demonstrate levels of necroptotic markers in TBS and triton fractions. a, Representative western blots of TB S and triton extracts from AD and CTL patients probed with the indicated antibodies. b-d, Quantitative analyses of the western blots. RIPK1 was not detected in the TBS fraction. For all the proteins measured, no changes were detected between the two groups. For RIPK1 triton [t(21)=0.840, p=0.409]; for RIPK3 TBS [t(21)=0.402, p=0.691]; for RIPK3 triton [t(21)=0.357, p=0.724]; for MLKL TBS [t(21)=0.176, p=0.862], for MLKL triton [t(21)=0.065, p=0.9491]. Data were normalized to (3-actin, used as a loading control. Data are presented as box plots and were analyzed by unpaired t-test. n=11 CTL cases and n=12 AD cases.

FIGS. 10a-10b are full blots of RIPK1 and MLKL in human cases. a-b, Proteins extracted with RIPA and UREA buffer from CTL and AD cases. Blots were probed with the indicated antibodies. The levels of the protein of interest (RIPK1 or MLKL) were normalized to (3-actin for every sample, then all samples were expressed as a ratio with respect to the average of the CTL samples in the same blot. Doing so, the AD samples are expressed as percentage change over the CTL samples.

FIGS. 11a-11e are full blots of RIPK1/MLKL immunoprecipitation and MLKL dimerization. a-b, Proteins from CTL and AD cases were immunoprecipitated with a RIPK1 antibody and probed with an MLKL antibody. The black arrows point to the MLKL band, the black arrowheads point to the IgG. c-e, Proteins from CTL and AD cases were run in not reducing conditions and probed with an MLKL antibody. The gray arrows point to the MLKL dimers, the gray arrowheads point to the MLKL monomers. The levels of MLKL dimers were normalized to the levels of MLKL monomers for every samples. Then all the samples in the same blot were expressed as ratio with respect to the average of the CTL sample in the same blot. Doing so, the AD samples are expressed as percentage change over the CTL samples.

FIGS. 12a-12d increased pMLKL in AD. a, Representative confocal images from CTL and AD cases stained with a different pMLKL antibody than the one used for FIG. 2. b, The graph shows the quantitative analyses of the pMLKL immunoreactivity tissue [t(28)=4.561; p<0.0001]. c, Representative confocal images from CTL and AD cases stained with the indicated antibodies. Statistical evaluation by Mander's correlation, followed by Costes randomization test indicates that in CTL cases, 36.04±1.5% of pMLKL immunoreactivity was located in the membrane [R(13)=0.319 and Costes p=0.96]. In AD cases, 52.90±3.2% of pMLKL immunoreactivity was located in the membrane [R(13)=0.365 and Costes p=0.97]. d, The graph shows the quantitative analyses of the colocalized pMLKL and cadherin pixels [t(28)=6.991; p<0.0001]. For all the data shown here, n=15 CTL cases and n=15 AD cases. Data in panels b and d were analyzed by unpaired t-test and are presented as box plots

FIGS. 13a-13b demonstrate that Caspase-3/pMLKL colocalization is similar between CTL and AD cases. a, Microphotographs of CTL (n=15 cases) and AD (n=15 cases) brains stained with the indicated antibodies. b, Quantitative analysis of the sections, which was obtained by Mander's correlation followed by Costes randomization test indicates that 42.10 ±1.9% of pMLKL immunoreactivity co-localized with Caspase-3 [R(13)=0.6159 and Costes p=0.965] for CTL, and 45.53±2.3% [R(13)=0.6075 and Costes p=0.970] for AD. There was no significant difference between the two groups [t(28)=0.319; p=0.752). Data are presented as box plots and were analyzed by unpaired t-test

FIGS. 14a-14c demonstrate that pMLKL is mainly found in neurons. a-c, Microphotographs of AD brain (n=15) sections co-labeled with the indicated antibodies. Statistical evaluation by Mander's correlation followed by Costes randomization test indicates that 60.22±3.3% of pMLKL immunoreactivity co-localized with NeuN [R(13)=0.583 and Costes p=0.99], 11.14±1.4% co-localized with GFAP [R(13)=0.179 and Costes p=0.63], and 28.00±2.6% co-localized with Ibal (R =0.578 and Costes p=0.96).

FIGS. 15a-15b demonstrate that RIPK1 and MLKL levels do not correlate with brain weight. a-b, Linear regression analysis without covariate correction between brain weight and RIPK3 or MLKL. The 95% confidence intervals is shown by the shaded area. No significant correlation was detected between RIPK3 or MLKL expression levels and brain weight. For RIPK3 [R(91)=0.002, p=0.986]; for MLKL [R(91)=-0.095, p=0.362]. n=93 AD cases. Data are presented as scatter blots.

FIG. 16 demonstrates that pMLKL co-localizes with phosphorylated tau. Sections from AD patients (n=15 cases) were stained with the indicated antibodies. Statistical evaluation by Mander's correlation followed by Costes randomization test indicates that 39.17±3.1% of pMLKL immunoreactivity co-localized with CP13 [R(13)=0.4155 and Costes p=0.99]. These data confirm the data shown in FIG. 4, using a different pMLKL antibody.

FIG. 17 is a schematic representation of an experimental design used to perform the causal inference testing. Gene regulatory network for RIPK1 based inferred from post mortem brain tissue samples. Causal inference testing was used to determine directed regulatory links between RIPK1 and its correlated genes.

FIGS. 18a-18f demonstrate levels of necroptotic markers in 5xFAD and APP/PS1 mice. a, Proteins from 5xFAD mice (n=5 mice) and littermate controls (NT; n=7 mice), and APP/PS1 mice (n=8) and littermate controls (WT; n=8), were probed with the indicated antibodies. b-d. Quantitative analyses of the blots show an increase of the kinases in the 5xFAD mice compared to control [t(10)=2.423, p=0.036 for RIPK1; t(10)=2.910, p=0.016 for MLKL; t(10)=3.076, p=0.012 for pMLKL]. No differences were found in the APP/PS1 mice compared to their control [t(14)=1.245, p=0.234 for RIPK1; t(14)=0.807, p=0.433 for MLKL]. e-f, Representative confocal microphotographs of sections from 5xFAD and APP/PS1 stained with Fluro-Jade. Quantification of western blots were obtained by normalizing the protein of interest to β-actin (used as a loading control). Data are presented as box plots and were analyzed by unpaired t-test.

FIGS. 19a-19f demonstrate that increasing necroptosis does not change AP pathology. a-b, Representative hippocampal sections from APP/PS1 mice injected with the AAV expressing GFP or MLKL. Sections were stained with an Aβ42-specific antibody. c-f, The graphs show soluble and insoluble Aβ40 and Aβ42 levels measured by sandwich ELISA. Panel c, t(26)=1.197, p=0.242. Panel d, t(26)=0.4513, p=0.655. Panel e, t(26)=0.6518, p=0.520. Panel f, t(26)=0.8277, p=0.415. Data are presented as box plots, and were analyzed by unpaired t-test. n=14 mice per group.

FIGS. 20a-20d demonstrate that increasing necroptosis does not change endogenous tau levels. a-b, Western blots of proteins extracted from APP/PS1-GFP (n=7 mice) and APP/PS1-MLKL mice (n=8 mice). Blots were probed with the indicated antibodies. The levels of Tau-5, which recognize total mouse tau, were similar between the two groups [t(13)=0.484; p=0.637]. The CP13 antibody, which is raised against tau phosphorylated at Ser202, recognized two bands of ˜60 and ˜50 kDa. Statistical analyses of both bands indicated that CP13 levels were similar between the two groups [t(13)=0.48; p=0.64] for both bands. Data are presented as box plots, and were analyzed by unpaired t-test.

FIGS. 21a-21b are full blots of proteins extracted from APP/PS1 and wild type primary neurons used for NeuN staining. Blots were probed with the indicated antibodies. Quantitative analyses are shown in FIGS. 8b-c.

FIGS. 22a-22b are full blots of proteins extracted from APP/PS1 and wild type primary neurons transfected with AAV-GFP eleven (DIV11) and fifteen (DIV) days after infection. Blots were probed with the indicated antibodies. Quantitative analyses are shown in FIGS. 8e-8f.

FIG. 23 are full blots of proteins extracted from 5xFAD and wild type mice. Blots were probed with the indicated antibodies. Quantitative analyses are shown in FIG. 8i. FIGS. 24a-24d are images of blots showing validation of the RIPK1, RIPK3, MLKL, and pMLKL antibodies. a, Proteins extracted from wild type cells, RIPK1 knockout cells (as a negative control), RIPK1 knockout cells transfected with a RIPK1 expressing plasmid (as a positive control), CTL and AD human cases, wild type and 5xFAD mice were probed with the RIPK1 antibody. The expected band of 73 kDa (arrow) was not present in the knockout cells. b, Proteins extracted from wild type mice, RIPK3 knockout mice (as a negative control), wild type cells transfected with a RIPK3 expressing plasmid (as a positive control), CTL and AD human cases, wild type and 5xFAD mice, were probed with the RIPK3 antibody. The expected band of 55 kDa (arrow) was not present in the RIPK3 knockout mice and was present in the cells transfected with the RIPK3-expressing plasmid. The RIPK3 band in the positive control ran a little slower as the plasmid had a GFP tag to its C-terminal. c, To validate the MLKL antibody, we loaded on a gel proteins extracted from wild type cells, MLKL knockout cells (as a negative control), MLKL knockout cells transfected with a MLKL-expressing plasmid (as a positive control), CTL and AD human cases, non-transgenic and 5xFAD mice. Notably, the expected band of 51 kDa (arrow) was not present in the knockout cells but it was present when these cells were transfected with a MLKL plasmid. d, To validate the phospho-specific MLKL antibody, we loaded on a gel protein extracted from MLKL knockout and wild type cells. As positive control, cells were treated with 1 ng/mL TNFa and 50 μM Caspase inhibitor zVAD-FMK for 4 hours prior protein extraction to induce necroptosis. These samples were used a positive controls.

FIGS. 25a-25b are images showing validation of the RIPK1, RIPK3, MLKL, and pMLKL antibodies. a, To validate the RIPK1 and MLKL antibodies, we used HAP1 cells (an immortalized human malignant neoplastic cell line) where the respective genes were knocked out. As control (WT) we used the parental cell line. To validate the RIPK3 antibody, we used mouse primary fibroblasts isolated from RIPK3 knockout and wild type mice. b, To validate the pMLKL antibody, we used HAP-1 cells and induced necroptosis activation as described in the methods.

While the invention is susceptible to various modifications and alternative forms, specific embodiments thereof have been shown by way of example in the drawings and are herein described in detail. It should be understood, however, that the description herein of specific embodiments is not intended to limit the invention to the particular forms disclosed, but on the contrary, the intention is to cover all modifications, equivalents, and alternatives falling within the spirit and scope of the invention as defined by the appended claims.

### DETAILED DESCRIPTION

Diagnostic and therapeutic methods for conditions associated with necroptosis and neuronal loss are provided. Necroptosis is a caspase-independent form of cell death in which programmed necrotic cell death is activated by inflammation or other microenvironmental factors (Linkermann et al., N Engl J Med. 2014; 370(5):455-465). In contrast to apoptosis, necroptosis does not appear to serve an important role in multicellular organism development, but instead plays a role in the defense against pathogens and is a likely culprit in many destructive inflammatory conditions. A key event in the activation of necroptosis is the formation of the necrosome, a filamentous amyloid-like complex made by Receptor Interacting Protein Kinase-1 (RIPK1) and Receptor Interacting Protein Kinase-3 (RIPK3). Upon formation of a highly regulated complex called the “necrosome,” necroptosis is executed by various pathways, including RIPK1's kinase activity and the activation of the pseudo-kinase Mixed Lineage Kinase Domain-Like (MLKL), which culminates in mitochondrial uncoupling and lipid peroxidation, and eventually cell death. Notably, elevated levels of RIPK3 and MLKL are routinely associated with an increase in necroptosis induction (Linkermann et al., N Engl J Med. 2014; 370(5):455-465).

The present disclosure is based at least in part on the inventor's discovery through biological analyses that levels of RIPK1 and MLKL1 are increased in human Alzheimer's disease (AD) brains compared to age-matched controls and that, in AD brains, RIPK1 and MLKL levels are positively correlated with brain weight and inversely correlated with the cognitive scores. Without wishing to be bound by theory, these data support a model in which necrosome formation, which is an invariable marker of necroptosis activation, is dependent on the physical interaction between RIPK1, RIPK3, and MLKL, and not on the relative levels of RIPK3.

In one aspect of the present disclosure, there is provided a method for reducing or inhibiting neuronal loss in a subject in need thereof, where the method comprises administering a therapeutically effective amount of a compound that inhibits production, activity, or expression of a protein associated with programmed necrotic cell death (e.g., “a necroptosis inhibitor”), whereby neuronal loss is reduced or inhibited in the subject. In some cases, the subject has or is suspected of having a disease associated with loss of neurons. As used herein, the term “neuronal loss” is used interchangeably with “loss of neurons,” and refers to the process of irreversible and functional loss of a neuron. During this process, neurons are damaged and can no longer exert their function of transmitting information by electrical and/or chemical signaling. The term “disease associated with a loss of neurons” refers to a neurological disorder wherein at least a symptom of such associated by or attributed to the loss of neurons manifests at some point in time during the disease. Symptoms may develop immediately after onset, for example after a major traumatic injury of the brain, but in some cases, such as in patients suffering from MS, symptoms may develop gradually. Herein, distinction is made between neurodegenerative diseases, which are associated with a progressive loss of neurons, typically as a result of subcellular processes and brain injuries or brain damage, which is mostly caused by physical damage.

In some cases, the necroptosis inhibitor reduces or inhibits production, activity, or expression of a RIP kinase (e.g., RIPK1, RIPK3) or MLKL. The necroptosis inhibitor can act directly or indirectly to reduce or inhibit said production, activity, or expression (e.g., mRNA expression, protein expression). In some cases, the compound reduces expression of at least one of RIPK1, RIPK3, or MLKL, or a fragment or variant thereof. In other cases, the necroptosis inhibitor acts directly or indirectly to reduce or inhibit the activity of at least one of RIPK1, RIPK3, or MLKL, or a fragment or variant thereof.

A necroptosis inhibitor as described herein may be any compound which performs the described function and thereby effects the inhibition of necroptosis. It will be understood that the compound is not limited to any particular chemotype, form, size, shape or conformation. Accordingly, the compound may be selected from the group consisting of synthetic compounds, organic synthetic drugs, small molecule organic drugs, natural small molecule compounds, other small molecule compounds, and peptides.

In certain embodiments, the necroptosis inhibitor comprises an RIPK1 enzyme inhibitor such as necrostatin-1 (Nec-1) or 7-Cl-O-necrostatin 1S (Nec-1S; (5-((7-chloro-1 H-indol-3-yl)methyl)-3-methylimidazolidine-2,4-dione)). It is shown herein that Nec-1S can be used to reduce or delay neuronal loss, such as losses associated with neurodegenerative disease. In some cases, the necroptosis inhibitor comprises a MLKL inhibitor such as necrosulfonamide ((E)-N-[4-[[(3-Methoxy-2-pyrazinyl)amino]sulfonyl]phenyl]-3-(5-nitro-2-thienyl)-2-propenamide), which selectively inhibits MLKL and, thus, blocks necrosis downstream of RIPK3 activation (Sun et al., 2012, Cell 148:213-27). In other cases, the necroptosis inhibitor is RIPK3 Inhibitor GSK'872 (N-(6-(Isopropylsulfonyl)quinolin-4-yl)benzo[d]thiazol-5-amine), which selectively inhibits RIPK3 (Kaiser et al., 2013, J. Biol. Chem. 288:31268).

The necroptosis inhibitor may comprise a small molecule, a microRNA (miRNA), a small interfering RNA (siRNA), or a short hairpin RNA (shRNA). The necroptosis inhibitor may be one that reduces production, activity, or expression of at least one of RIPK1, RIPK3, or MLKL, or a fragment or variant thereof.

In certain embodiments, the necroptosis inhibitor is an agent that inhibits phosphorylation of RIPK1, MLKL, or RIPK3, thereby blocking kinase activity of the protein. For example, the necroptosis inhibitor can be an agent that directly or indirectly inhibits phosphorylation of MLKL at an amino acid position selected from the group consisting of position 357 and position 358, wherein the position is numbered relative to an MLKL (e.g., the MLKL amino acid sequence encoded by NCBI Reference Sequence NM_{'}152649). The nucleotide sequence of NCBI Reference Sequence NM_152649 is set forth herein as SEQ ID NO:1, and the amino acid encoded by NCBI Reference Sequence NM_152649 is set forth as SEQ ID NO:2.

In some cases, the necroptosis inhibitor is an agent that directly or indirectly modulates necroptosis activation. The agent may modulate the expression or the activity of any component (e.g., protein, enzyme, kinase) in the pathway leading to necroptosis activation. In some cases, the agent directly or indirectly acts on a component which is able to affect (act on) a RIP kinase (e.g., RIPK1, RIPK3) or MLKL. The agent typically has an activity which directly or indirectly (e. g. mediated through a components as discussed above) results in an effect on necroptosis activation which is generally counter (opposite) to the effect of aberrant necroptosis activation which causes neuronal loss. As used herein, the term “modulate” includes any of the ways mentioned herein in which the agent of the invention is able to modulate a component to affect neuronal loss and/or necroptosis activation. Whether or not a candidate agent directly or indirectly modulates the activity of a RIP kinase (e.g., RIPK1, RIPK3) or MLKL can be determined by performing an assay to detect changes in kinase activity in the presence and absence of the candidate agent.

The terms “treating”, “treatment” and “therapy” are used herein to refer to curative therapy, prophylactic therapy, and preventative therapy. The terms embrace both preventative, i.e., prophylactic, and palliative treatments. Thus, in the context of the present disclosure the term “treating” encompasses curing, ameliorating or tempering the severity of neuronal loss, necroptosis, and/or associated diseases or their symptoms. In some cases, the term “treated” refers to any beneficial effect on progression of a disease or condition. Beneficial effects can include reversing, alleviating, inhibiting the progress of, preventing, or reducing the likelihood of the disease or condition to which the term applies or one or more symptoms or manifestations of such a disease or condition. “Preventing” or “prevention” means preventing the occurrence of the necroptosis or tempering the severity of the necroptosis if it develops subsequent to the administration of the compounds or pharmaceutical compositions of the present invention. The term “inhibit” is used to describe any form of inhibition that results in prevention, reduction or otherwise amelioration of neuronal loss associated with necroptosis. As described herein, inhibition of neuronal loss includes both complete and partial inhibition of neuronal loss or necroptosis. In one embodiment, inhibition is complete inhibition. In another embodiment, inhibition is partial inhibition.

In the context of this disclosure, the term “administering” and variations of that term including “administer” and “administration”, includes contacting, applying, delivering or providing a compound or composition of the invention to a subject by any appropriate means. For example, in the context of administering an agent that is an inhibitor of neuronal loss or necroptosis activation to a subject, an effective amount of an agent is, for example, an amount sufficient to achieve a reduction in neuronal loss as compared to the response obtained without administration of the agent.

By “subject” or “individual” or “animal” or “patient” or “mammal,” is meant any subject, particularly a mammalian subject, for whom diagnosis, prognosis, or therapy is desired. Mammalian subjects include humans, domestic animals, farm animals, and zoo, sport, or pet animals such as dogs, cats, guinea pigs, rabbits, rats, mice, horses, cattle, cows, and so on. Thus, in addition to being useful for human treatment (e.g., treating a human patient), the compounds of the present invention may also be useful for veterinary treatment of mammals, including companion animals and farm animals.

The methods disclosed herein can be used to treat conditions (e.g., disorders or diseases) associated with neuronal loss and in which necroptosis is likely to play a substantial role (e.g., conditions associated with aberrant necroptosis activation). Such conditions include, without limitation, neurodegenerative diseases of the central or peripheral nervous system and muscular dystrophies and related diseases including muscle wasting diseases. Exemplary neurodegenerative diseases are Alzheimer's disease (AD), Huntington disease (HD), Parkinson's disease (PD), amyotrophic lateral sclerosis (ALS), Down Syndrome, HIV-associated dementia, cerebral ischemia, multiple sclerosis, Lewy body disease, Menke's disease, Wilson's disease, Creutzfeldt-Jakob disease, and Fahr disease. Exemplary muscular dystrophies or related diseases are Becker's muscular dystrophy, Duchenne muscular dystrophy, myotonic dystrophy, limb-girdle muscular dystrophy, Landouzy-Dejerine muscular dystrophy, facioscapulohumeral muscular dystrophy (Steinert's disease), myotonia congenita, Thomsen's disease, and Pompe's disease. Muscle wasting can be associated with cancer, AIDS, congestive heart failure, and chronic obstructive pulmonary disease, as well as include necrotizing myopathy of intensive care. In some cases, the condition associated with neuronal loss and/or aberrant necroptosis activation is a condition in which necroptosis is likely to play a substantial role, including, but not limited to those described herein.

In some cases, the condition in which necroptosis is likely to play a substantial role is one resulting from retinal neuronal cell death; cell death of cardiac muscle; cell death of cells of the immune system; cell death associated with renal failure; stroke; liver disease; pancreatic disease; heart, mesenteric, retinal, hepatic or brain ischemic injury; ischemic injury during organ storage; head trauma; septic shock; coronary heart disease; cardiomyopathy; bone avascular necrosis; sickle cell disease; muscle wasting; gastrointestinal disease; tuberculosis; diabetes; alteration of blood vessels; muscular dystrophy; graft-versus-host disease; viral infection (e.g., acute, latent, and persistent viral infections); bacterial infection; Crohn's disease; ulcerative colitis; asthma; atherosclerosis; pain (e.g., inflammatory pain, diabetic pain, or pain associated from trauma or burn); chronic or acute inflammatory conditions such as rheumatoid arthritis, psoriasis, and Stevens-Johnson syndrome; and conditions in which an alteration in cell proliferation, differentiation, or intracellular signaling is a causative factor (e.g., cancer, microbial infection).

Pharmaceutical compositions may be formulated from compounds described herein for any appropriate route of administration including, for example, topical (for example, transdermal or ocular), oral, buccal, nasal, or parenteral administration. The term parenteral as used herein includes subcutaneous, intradermal, intravascular (for example, intravenous), intramuscular, spinal, intracranial, intrathecal, intraocular, periocular, intraorbital, intrasynovial and intraperitoneal injection, as well as any similar injection or infusion technique. In certain embodiments, compositions in a form suitable for oral use or parenteral use are preferred. Suitable oral forms include, for example, tablets, troches, lozenges, aqueous or oily suspensions, dispersible powders or granules, emulsions, hard or soft capsules, or syrups or elixirs. For intravenous, intramuscular, subcutaneous, or intraperitoneal administration, one or more compounds may be combined with DMSO. Such formulations may be prepared by dissolving solid active ingredient in DMSO and in some cases, one or more physiologically compatible substances such as water, sodium chloride, or glycine, and having a buffered pH compatible with physiological conditions to produce an aqueous solution, and rendering said solution sterile. The formulations may be present in unit or multi-dose containers such as sealed ampoules or vials.

Examples of components are described in Martindale—The Extra Pharmacopoeia (Pharmaceutical Press, London 1993) and Martin (ed.), Remington's Pharmaceutical Sciences.

In some cases, compounds disclosed herein are be evaluated for their pharmacological properties in animal models of disease. The compounds identified to decrease neuronal loss may be structurally modified and subsequently used to decrease neuronal loss, or to treat a subject with a condition associated with neuronal loss and/or aberrant necroptosis. The methods used to generate structural derivatives of the small molecules that decrease neuronal loss are readily known to those skilled in the fields of organic and medicinal chemistry.

Regardless of the route of administration selected, therapeutic agents of the present invention, which may be used in a suitable hydrated form, and/or the pharmaceutical compositions of the present invention, can be formulated into pharmaceutically acceptable dosage forms by conventional methods known to those of skill in the art. For the inhibition of neuronal loss, the dose of the biologically active compound according to the invention may vary within wide limits and may be adjusted to individual requirements. It will be appreciated that different dosages may be required for treating different disorders. As used herein, the terms “therapeutically effective amount” or “effective amount” refer to an amount of necroptosis inhibitor that results in an improvement or remediation of the symptoms of necrosis, necroptosis, and/or associated diseases or their symptoms. For example, an effective amount of an agent is that amount which causes a statistically significant decrease in necroptosis. One of skill in the art in possession of this disclosure can optimize the dosages as appropriate.

A therapeutically effective dose relates to the amount of a compound which is sufficient to improve the symptoms, for example a treatment, healing, prevention or improvement of such conditions. In exemplary embodiments, a therapeutically effective amount or dose is an amount such that free antibody is present in the blood. For dosage determinations, it can be advantageous to assess toxicity and therapeutic efficacy of a compound in cell cultures or in experimental animals. For example, the LD₅₀ (i.e., the dose lethal to 50% of the population) and ED₅₀ (i.e., the dose therapeutically effective in 50% of the population) can be determined. From these calculations, dosage ranges for use in humans can be formulated. Dosage ranges can vary depending on factors such as mode of administration. A therapeutically effective amount of a pharmaceutical composition provided herein can range from about 0.001 to 100 mg of antibody per kg body weight of the subject (e.g., about 0.01 to 100 mg/kg body weight; about 0.1 to 40 mg/kg body weight; about 1 to 20 mg/kg body weight).

In some cases, an appropriate dose for a necroptosis inhibitor can be from 0.005 mg/kg up to a maximum tolerated dose. In some cases, an appropriate dose of a pharmaceutical composition as provided herein can be determined according to body surface area of a subject, calculated using the subject's height and weight, to whom the composition will be administered. In such cases, a dose can be provided as a particular amount of the composition per m² (e.g., mg/m²). In some cases, an appropriate dose can be between approximately 10 mg/m² and approximately 40 mg/m² of a monoclonal antibody. When converted to milligrams (mg) per kilogram (kg) of a subject's body weight, a dose of 15 mg/m² is the same as about 0.4 mg/kg. Additional information about dosage calculation can be found in Center for Drug Evaluation and Research, Center for Biologics Evaluation and Research (2002), Estimating the safe starting dose in clinical trials for therapeutics in adult healthy volunteers, U.S. Food and Drug Administration, Rockville, Md., USA.

It will be understood that mass of pharmaceutical composition comprising a monoclonal antibody as provided herein can refer to mass of the antibody plus a delivery agent or pharmaceutically acceptable carrier, if applicable. In some cases, dosages and dosage ranges appropriate for a composition provided herein can be determined using pharmacokinetic data (i.e., drug metabolism and clearance). As used herein, “pharmacokinetics” refers to the process by which a drug or pharmaceutical composition is absorbed, distributed, metabolized, and excreted from the body.

Clinicians, physicians, and other health care professionals can administer a composition to a subject in need thereof according to a method provided herein by a physician or other health professional. In some cases, a single administration of the composition may be sufficient. In other cases, more than one administration of the composition is performed at various intervals (e.g., once per week, twice per week, daily, monthly) or according to any other appropriate treatment regimen. The duration of treatment can be a single dose or periodic multiple doses for as long as administration of a composition provided herein is tolerated by the subject.

Any appropriate method can be practiced to determine, detect, or monitor a subject's response to treatment according to a method provided herein. As used herein, “determining a subject's response to treatment” refers to the assessment of the results of a therapy in a subject in response to administration of a composition provided herein or to treatment according to a method provided herein. For example, a subject's condition can be monitored continuously or evaluated at appropriate time intervals (e.g., at regular or irregular time points) to detect and/or monitor any changes in disease progression (e.g., change or stabilization of cognitive function or motor functions) as an indicator of the subject's response to administration of a necroptosis inhibitor according to a method described herein. In some cases, detection methods such as computed tomography (CT), magnetic resonance imaging (MM), and positron emission tomography (PET) can be used. In some cases, evaluation of a subject's response to treatment as provided herein can include scoring the subject's cognitive function, preferably prior to and following treatment according to a method provided herein. Any appropriate functional assessment scoring system can be used to assess cognitive loss. See, for review, Baldwin et al., Curr. Protoc. Neurosci. 0 10:Unit10.3, 2009;

In another aspect, provided herein are methods for detecting aberrant necroptosis activation in a subject. In some cases, the methods comprise detecting differential expression of at least one necroptosis marker in a biological sample from the subject relative to a control biological sample. For example, differential expression of at least one biomarker selected from the group consisting of RIPK1 and MLKL relative to a level of expression of the one or more biomarker in the control biological sample indicates that the subject has a condition associated with aberrant necroptosis activation (e.g., Alzheimer's disease (AD), multiple sclerosis (MS), Huntington's Disease (HD), Parkinson's Disease (PD), and amyotrophic lateral sclerosis (ALS)). Exemplary biomarkers for such methods include, for example, RIPK1 mRNA, a MLKL mRNA, phosphorylated MLKL (pMLKL) protein, MLKL protein, and RIPK1 protein.

In a further aspect, provided herein is a method of diagnosing neurodegenerative disease in a subject. In some cases, the method comprises detecting differential expression of at least one biomarker selected from the group consisting of RIPK1 and MLKL in a biological sample from the subject relative to expression of the at least one marker in a control biological sample obtained from a subject free of neurodegenerative disease. Differential expression of the least one biomarker relative to a level of expression of the at least one biomarker in the control biological sample indicates that the subject has a neurodegenerative disease. Exemplary biomarkers for such methods include, for example, RIPK1 mRNA, a MLKL mRNA, phosphorylated MLKL (pMLKL) protein, MLKL protein, and RIPK1 protein. The biological sample can be a tissue sample, body fluid sample (e.g., blood, cerebral spinal fluid), biopsied tissue, or any other sample obtained from the body of the subject.

In another aspect, compounds described herein can also be used in screening methods to identify targets of necroptosis and to identify additional inhibitors of necroptosis, as well as in assay development. Accordingly, in one embodiment the present disclosure provides a screening method as described herein which further comprises testing the ability of the candidate compound to rescue cell death by necroptosis. In some cases, the screening method comprises using RIPK1 as a target, and candidate compounds are assayed for their ability to bind to or otherwise inhibit RIPK1. For example, assays that measure inhibition of autophosphorylation of RIP1 can be used. Alternatively, assays that measure binding of a candidate compound to RIPK1 are useful in the methods of the invention. In other cases, the screening method comprises using MLKL as a target, and candidate compounds are assayed for their ability to bind to or otherwise inhibit MLKL. For example, assays that measure inhibition of autophosphorylation of MLKL can be used. Alternatively, assays that measure binding of a candidate compound to MLKL are useful. Other variations of binding assays are known in the art and can be employed. For example, RIPK1 binding assays are described, e.g., in U.S. Pat. No. 6,211,337, which is hereby incorporated by reference.

To identify compounds that are selective or specific for RIPK1 or MLKL, screening assays can be performed using multiple targets. For example, for a given candidate compound, the binding, autophosphorylation, or other measure of target activity may be assayed for both RIPK1 and MLKL, or alternatively both RIPK1 and RIPK3 or both RIPK3 and MLKL, and the results compared. Candidate compounds that exert a greater effect on RIPK1 than RIPK3, or another homologue or other molecule chosen for this purpose, are considered to be specific for RIPK1, and may be particularly desirable in the methods of the invention. Other assays are known in the art, and any method for measuring protein interactions or inhibition of the activity of a target molecule (e.g., RIPK1 or MLKL) may be utilized. Such methods include, but are not limited to fluorescence polarization assays, mass spectrometry (Nelson and Krone, J. Mol. Recognit., 12:77-93, 1999), surface plasmon resonance (Spiga et al., FEBS Lett., 511:33-35, 2002; Rich and Mizka, J. Mol. Recognit., 14:223-228, 2001; Abrantes et al., Anal. Chem., 73:2828-2835, 2001), fluorescence resonance energy transfer (FRET) (Bader et al., J. Biomol. Screen, 6:255-264, 2001; Song et al., Anal. Biochem. 291:133-41, 2001; Brockhoff et al., Cytometry, 44:338-248, 2001), bioluminescence resonance energy transfer (BRET) (Angers et al., Proc. Natl. Acad. Sci. USA, 97:3684-3689, 2000; Xu et al., Proc. Natl. Acad. Sci. USA, 96:151-156, 1999), fluorescence quenching (Engelborghs, Spectrochim. Acta A. Mol. Biomol. Spectrosc., 57:2255-2270, 1999; Geoghegan et al., Bioconjug. Chem. 11:71-77, 2000), fluorescence activated cell scanning/sorting (Barth et al., J. Mol. Biol., 301:751-757, 2000), ELISA, and radioimmunoassay (RIA).

In another embodiment, provided herein are screening methods in which the method tests the ability of a candidate compound to reduce or inhibit neuronal loss. In some cases, the testing is performed in an in vitro cell based assay.

The articles “a” and “an” are used herein to refer to one or to more than one (i.e., to at least one) of the grammatical object of the article. By way of example, “an antibody” means one antibody or more than one antibody. As such, the terms “a” (or “an”), “one or more,” and “at least one” are used interchangeably herein.

While the present invention is susceptible to various modifications and alternative forms, specific embodiments thereof have been shown by way of example in the drawings and are herein described in detail. It should be understood, however, that the description herein of specific embodiments is not intended to limit the invention to the particular forms disclosed. To the contrary, it should be appreciated that many equivalents, alternatives, variations, and modifications, aside from those expressly stated, are possible and fall within the spirit and scope of the invention as defined by the appended claims.

The invention will be more fully understood upon consideration of the following non-limiting Examples.

### EXAMPLE

In this Example, we demonstrate that necroptosis is activated in postmortem human AD brains, where it positively correlates with Braak stage (meaning, the stage in the Alzheimer's disease continuum of progressive brain deterioration) and inversely correlates with brain weight and cognitive scores. Notably, in a gene regulatory network built from post-mortem brain tissue, the set of genes regulated by RIPK1 overlaps significantly with multiple, independent AD transcriptomic signatures, indicating that RIPK1 activity could explain a significant portion of described transcriptomic changes in AD. We further demonstrate that reducing necroptosis activation with a RIPK1 inhibitor reduces cell loss in 5xFAD mice, a mouse model of AD. Together, these data provide compelling evidence that necroptosis is activated in AD and thus may contribute to neurodegeneration in this insidious disorder.

Results

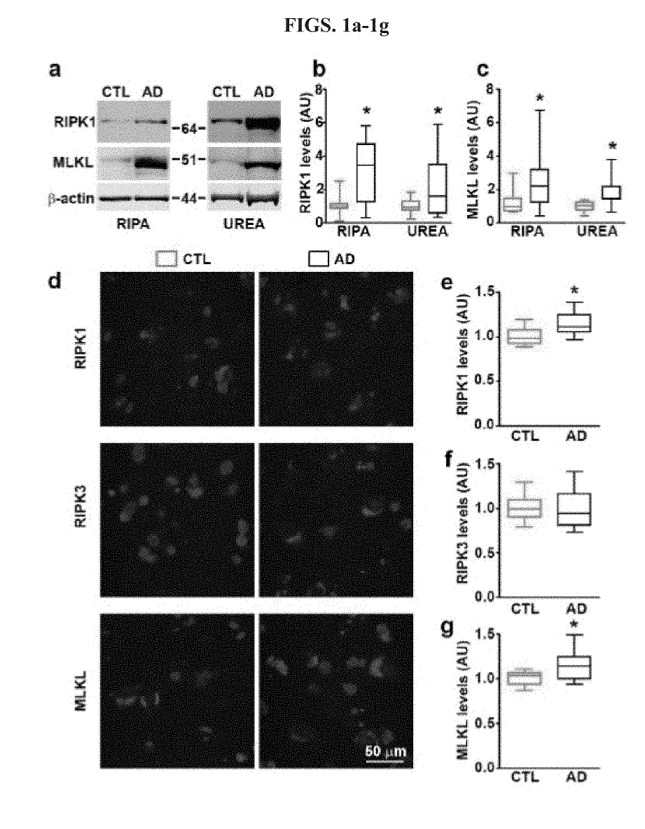

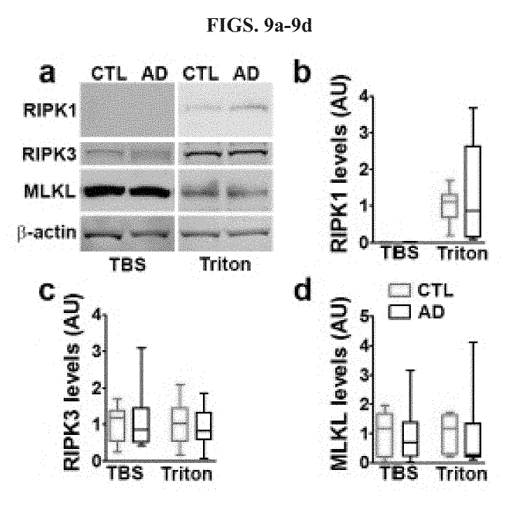

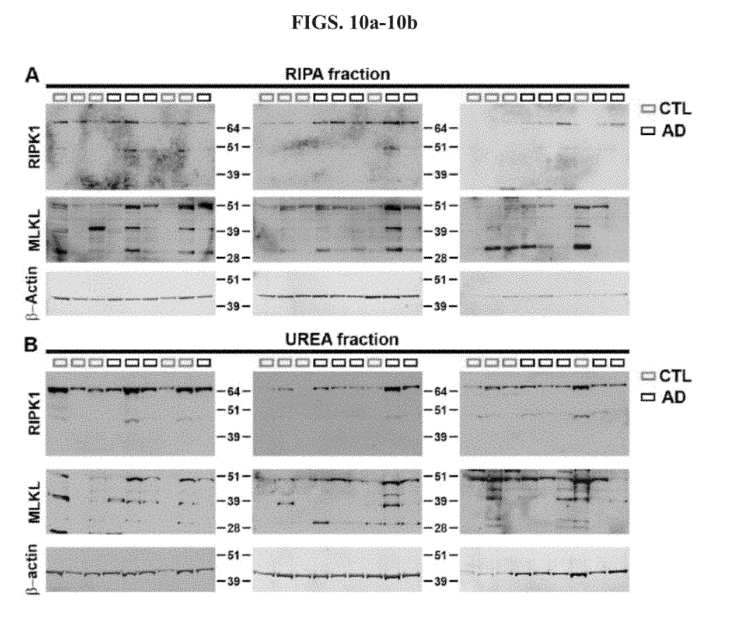

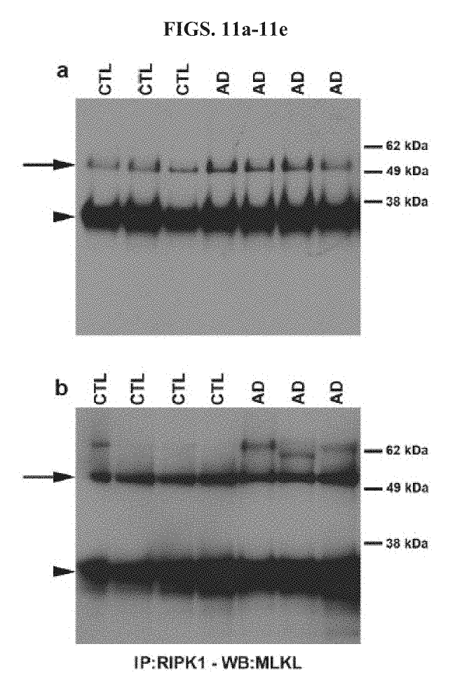

Necroptosis is activated in human AD brains: we measured RIPK1, RIPK3, and MLKL in the temporal gyms of AD (n=12) and CTL cases (n=11) using buffers of increasing stringency (see Table 1 for case information). We found that RIPK1 was not detectable in the TBS fraction and was not statistically significant between CTL and AD brains in the triton fraction (FIGS. 9a-9b). Similarly, RIPK3 and MLKL levels in the TBS and triton fractions were similar between AD and CTL cases (FIGS. 9a, 9c-9d). In contrast, in RIPA and urea fractions, RIPK1 and MLKL levels were significantly higher in AD compared to CTL cases (FIGS. 1a-1c), while RIPK3 was not detectable (full blots are shown in FIG. 10). These observations are consistent with previous reports showing that necroptotic markers, once activated, form insoluble amyloid-like structures¹⁶. The changes in solubility of the necrosome have been also confirmed in the cortex of people with multiple sclerosis¹³. We also measured the levels of RIPK1, RIPK3, and MLKL by immunofluorescence. Consistent with the biochemical data, we found that RIPK1 and MLKL immunoreactivity was higher in AD compared to CTL cases. In contrast, RIPK3 levels were similar between the two groups (FIGS. 1d-1g). Taken together, these data indicate that the steady-state levels of two key proteins involved in necroptosis activation are increased in postmortem human AD brains. The changes in MLKL levels are most notable as, independent of the mechanisms that trigger necroptosis, MLKL activation is the most proximal step to membrane disruption and cell death^{9,10}.

TABLE 1
Demographic characteristics and ApoE genotype distribution for cases (AD) and controls
(CTL) used in the western blot and immunofluorescence analysis
Age distribution

| | | | | 25% | 50% | 75% | | |
|---|---|---|---|---|---|---|---|---|
| Status (n) | Mean | s | Min | ile | ile | ile | Max | P* |
| AD (30) | 82.60 | 7.60 | 64.00 | 77.75 | 85.00 | 88.25 | 96.00 | 0.438 |
| CTL (28) | 84.07 | 6.81 | 71.00 | 78.25 | 84.50 | 88.75 | 97.00 | |

Gender distribution

| Status (n) | M (%) | F (%) | P** |
|---|---|---|---|
| AD (30) | 16 (53.3) | 14 (46.7) | 1.000 |
| CTL (28) | 15 (53.6) | 13 (46.4) | |

ApoE genotype distribution (%)

| Status (n) | 2/2 | 2/3 | 2/4 | 3/3 | 3/4 | 4/4 | P** |
|---|---|---|---|---|---|---|---|
| AD (30) | 0 (0) | 3 (10) | 0 (0) | 13 (43.3) | 11 (36.7) | 3 (10) | 5.00E−05 |
| CTL (28) | 1 (3.6) | 3 (10.7) | 0 (0) | 18 (64.3) | 5 (17.8) | 0 (0) | |

ApoE4 carrier distribution (%)

| Status (n) | E4+ | E4− | P** |
|---|---|---|---|
| AD (30) | 14 | 16 | 2.60E−02 |
| CTL (28) | 5 | 23 | |

For Table 1: The groups were homogenous for age and gender distributions. Predictably, ApoE genotype and E4 carrier distributions were statistically different between CTL and AD. *t-test equal variance. **Fisher's exact test (20,000 simulated P values).

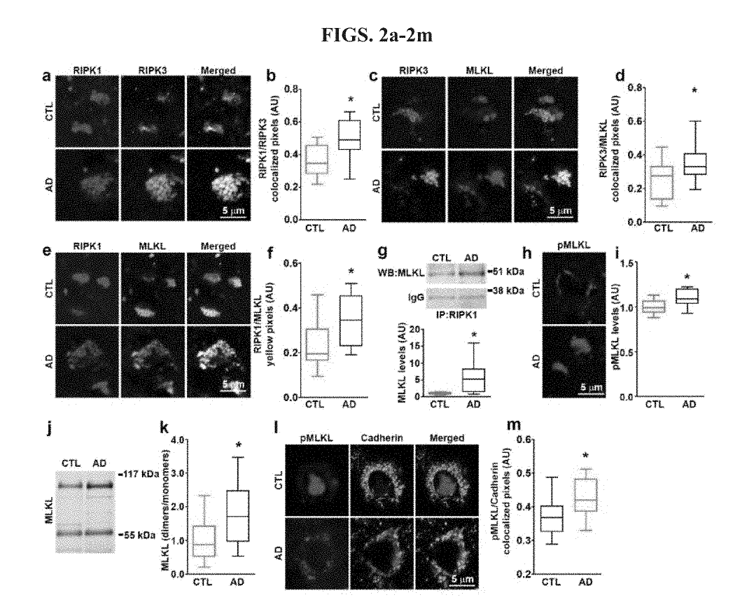

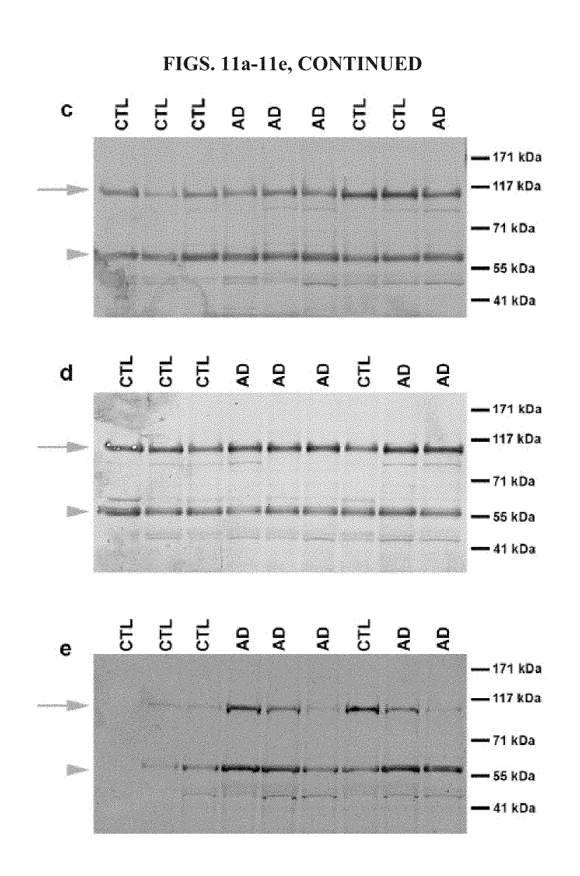

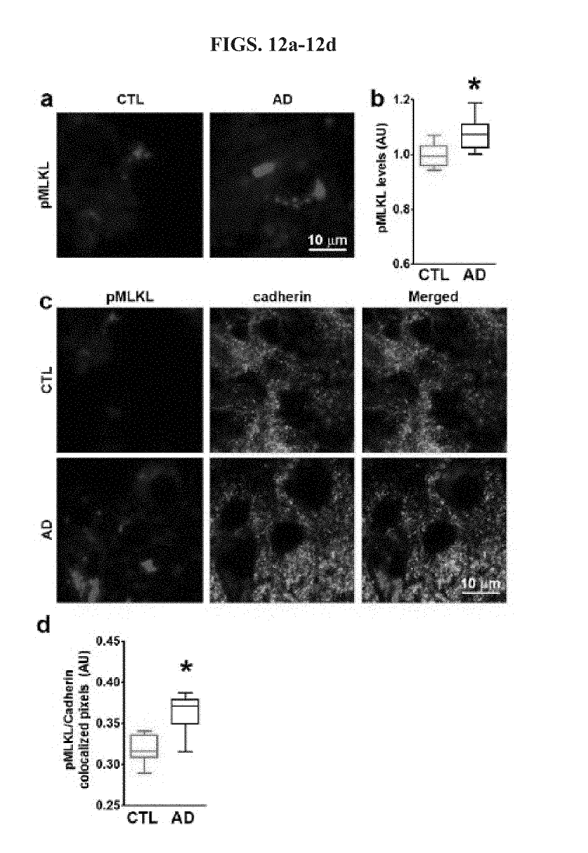

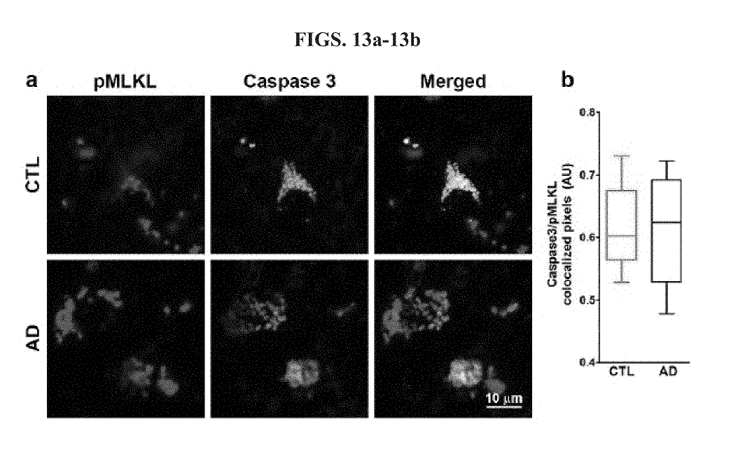

During necroptosis activation, RIPK1 binds to and activates RIPK3. In turn, activated RIPK3 binds to and activates MLKL^{9, 10}. To determine whether these events occur in human AD brains, we co-labeled hippocampus and temporal gyms sections from human AD and CTL cases with RIPK1 and RIPK3 antibodies. Confocal imaging indicated a significantly higher degree of co-localization between RIPK1 and RIPK3 in human AD brains compared to CTL cases (FIGS. 2a-2b). Similarly, we found a 41.9±12.6% increase in the co-localization between RIPK3 and MLKL (FIGS. 2c-2d), and a 41.8±14.5% increase in co-localization between RIPK1 and MLKL (FIGS. 2e-2f). To further confirm an interaction between RIPK1 and MLKL, we performed co-immunoprecipitation experiments. Specifically, we used a RIPK1 antibody to pull down RIPK1 and its binding partners. Western blot analyses of the pulled down products indicated a stronger MLKL-RIPK1 interaction in AD brains compared to CTL cases (FIG. 2g; full blots are shown in FIGS. 11a-11b). Necroptosis is executed by phosphorylated MLKL at Thr357/Ser358 (pMLKL). Once phosphorylated, pMLKL aggregates to form homodimers, which induce membrane damage leading to cell loss^{9, 10}. Using confocal imaging, we found a significant increase in pMLKL levels in human AD brains compared to CTL cases (FIGS. 2h-2i). Consistent with these findings, MLKL dimers were significantly increased in AD brains (FIGS. 2j-2k; full blots are shown in FIGS. 11c-11e). Further, we assessed the amount of pMLKL in AD brains that co-localizes with the membrane marker cadherin and found that 42.31±2.0% of pMLKL immunoreactivity was localized to the membrane (FIGS. 2l-2m). Statistical evaluation indicated that the Pearson's correlation coefficient was R=0.630 with a Costes randomization p value of 0.980, which indicate that there was 98% likelihood that the co-localization is not random¹⁷. We confirmed the increase in pMLKL in AD brains and its co-localization with cadherin using a second, different pMLKL antibody (FIG. 12). To determine whether neurons undergoing necroptosis also show markers of apoptosis, we double stained sections with pMLKL and a caspase-3 antibody (FIG. 13a). We found that the probability of finding co-localization between pMLKL and caspase-3 was not statistically significant between AD and CTL cases (p>0.05; FIG. 13b). Taken together, these data provide the first direct evidence of necrosome formation in human AD brains. This is notable, as overwhelming evidence indicates that necrosome formation is sufficient and necessary for necroptosis activation and thus cell death¹⁰.

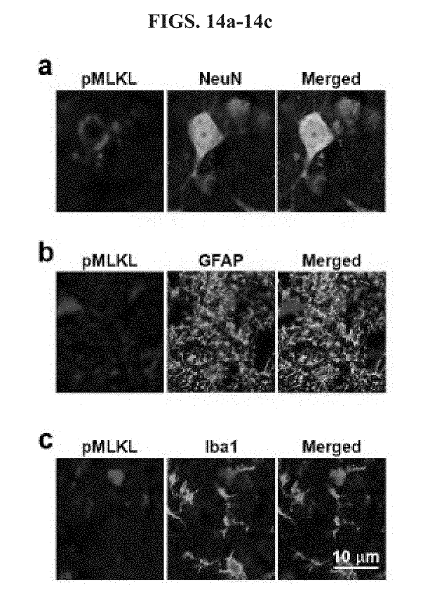

To determine the cell type in which pMLKL is most active, we co-labeled human AD brains with pMLKL and neuronal, astrocytic or microglia markers. We found that 60.22±3.3% of pMLKL immunoreactivity co-localized with NeuN, 11.14±1.4% co-localized with GFAP, and 28.00±2.6% co-localized with Ibal (FIGS. 14a-14c). These data suggest that most of the pMLKL is found in neurons and microglia.

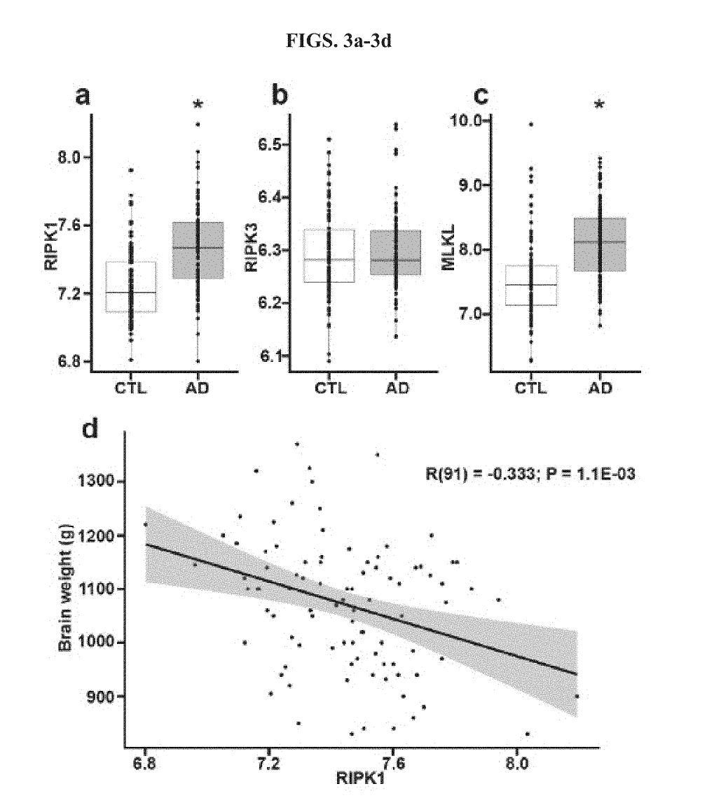

Necroptosis activation negatively correlates with brain weight and MUSE: To further corroborate our findings, we leveraged an unbiased, genome-wide mRNA screening between AD (n=97) and CTL cases (n=98) using microarray technology (see Tables 2-4 for cases information and variables distributions). After filtering out low expressed probes, we obtained a final dataset of 26,583 transcripts. We detected one probe mapping to RIPK1 (NM 003804), one probe mapping to RIPK3 (NM_006871), and three probes mapping to MLKL, corresponding to 2 different transcript variants (NM_152649 and XM_001126647). While RIPK3 mRNA levels were not significantly different between the two groups, we found RIPK1 and MLKL mRNA levels were significantly higher in human AD brains, both at the nominal and genome-wide level (FIGS. 3a-3c and Table 5). Among the significant probes mapping to MLKL, we focused on the transcript NM_152649, since it showed a higher average expression and bigger change in expression levels than XM_001126647. To validate these RNA-expression analyses, we used a publicly available, independent data set (GSE5281; n=12 AD cases and n=6 CTL cases). Notably, this data set has been generated from laser-captured neurons from medial temporal gyrus^{18, 19}. We found that RIPK1 and MLKL levels were increased in AD cases compared to controls (Table 6). These changes were statistically significant at a genome-wide level (correction for 20,514 tests; adj-p=0.023 and 0.005 for RIPK1 and MLKL, respectively). In contrast, RIPK3 levels were similar between the two groups (Table 6). These data confirm that necroptotic markers are upregulated in postmortem human AD cases, and also are consistent with the data shown in FIG. 14a, indicating that most of the pMLKL in AD co-localizes with neurons.

TABLE 2
Demographic characteristics and ApoE genotype distribution for cases (AD) and
controls (CTL) used in the expression profiling analysis.
Age distribution

| Status (n) | mean | s | Min | 25% ile | 50% ile | 75% ile | Max | P* |
|---|---|---|---|---|---|---|---|---|
| AD (97) | 85.02 | 6.75 | 70.00 | 81.00 | 86.00 | 90.00 | 98.00 | 1.000 |
| CTL (98) | 84.98 | 6.90 | 70.00 | 80.25 | 85.00 | 90.00 | 102.00 | |

Gender distribution

| Status (n) | M (%) | F (%) | P** |
|---|---|---|---|
| AD (97) | 49 (51.5) | 48 (49.5) | 1.000 |
| CTL (98) | 50 (51.0) | 48 (49.0) | |

Apo E genotype distribution (%)

| Status (n) | 2/2 | 2/3 | 2/4 | 3/3 | 3/4 | 4/4 | P |
|---|---|---|---|---|---|---|---|
| AD (97) | 0 (0.0) | 4 (4.1) | 1 (1.0) | 39 (40.2) | 42 (43.3) | 11 (11.3) | 1.69E−05 |
| CTL (95) | 3 (3.2) | 19 (20.0) | 1 (1.1) | 48 (50.5) | 22 (23.2) | 2 (2.1) | |

Apo E4 carrier distribution (%)

| Status (n) | E4+ | E4− | P** |
|---|---|---|---|
| AD (97) | 54 (55.7) | 43 (44.3) | 3.95E−05 |
| CTL (95) | 25 (26.3) | 70 (73.7) | |

TABLE 3
Descriptive statistics for sample characteristics and continuous variables in AD
and CTL cases.

| | | Total | | | | | | | | | |
|---|---|---|---|---|---|---|---|---|---|---|---|
| Variable | Type | n | | mean | | s | | Range | | P (Sh) | |
| Height (m) | C | 127 | | 1.67 | | 0.10 | | 1.42-1.89 | | 0.021 | |
| Weight (Kg) | C | 132 | | 65.92 | | 15.60 | | 35.8-112 | | 0.062 | |
| BMI | C | 127 | | 23.46 | | 4.390 | | 15.5-34.57 | | 0.133 | |
| Brain Weight (g) | C | 190 | | 1130.5 | | 131.9 | | 830-1560 | | 0.232 | |
| Expired Age (years) | C | 191 | | 84.81 | | 6.8 | | 70-102 | | 0.137 | |
| MMSE* | I | 113 | | 19.35 | | 10.55 | | 0-30 | | 6.89−E10 | |
| | AD | | | | | CTL | | | | | |
| Variable | n | mean | s | Range | P (Sh) | n | mean | s | Range | P (Sh) | P |
| Height (m) | 64 | 1.67 | 0.10 | 1.47-1.89 | 0.054 | 63 | 1.67 | 0.10 | 1.42-1.89 | 0.581 | 0.817 |
| Weight (Kg) | 66 | 65.74 | 13.9 | 35.8-92.5 | 0.467 | 66 | 66.09 | 17.19 | 39.9-112.0 | 0.020 | 0.736 |
| BMI | 64 | 23.60 | 4.13 | 15.50-32.37 | 0.296 | 63 | 23.31 | 4.66 | 15.71-34.57 | 0.075 | 0.707 |
| Brain Weight (g) | 93 | 1071.29 | 123.3 | 830-1370 | 0.335 | 97 | 1187.32 | 114.21 | 890-1560 | 0.393 | 1.96E−10 |
| Expired Age (ys) | 94 | 84.64 | 6.69 | 70-98 | 0.304 | 97 | 84.97 | 6.93 | 70-102 | 0.705 | 0.730 |
| MMSE* | 62 | 12.14 | 9.21 | 0-28 | 0.00021 | 51 | 28.12 | 1.76 | 24-30 | 6.08E-05 | 2.20E−16 |

TABLE 4
Distribution of ordinal variables in total (TOT), AD, and CTL samples.

| | | TOT | AD | CTL | |
|---|---|---|---|---|---|
| | Class | n = 191 | n = 94 | n = 97 | P |
| Braak Stage | I | 17 (8.9) | 0 (0.0) | 17 (17.5) | 0.0014 |
| | II | 29 (15.2) | 7 (7.4) | 22 (22.7) | |
| | III | 43 (22.5) | 6 (6.4) | 37 (38.1) | |
| | IV | 35 (18.3) | 14 (14.9) | 21 (21.6) | |
| | V | 40 (20.9) | 40 (42.6) | 0 (0.0) | |
| | VI | 27 (14.1) | 27 (28.7) | 0 (0.0) | |
| Plaque | Zero | 33 (17.3) | 0 (0.0) | 33 (34.0) | 5.00E−05 |
| Density | Sparse | 32 (16.8) | 1 (1.1) | 31 (32.0) | |
| | Moderate | 46 (24.1) | 20 (21.3) | 26 (26.8) | |
| | Frequent | 80 (41.9) | 73 (77.7) | 7 (7.2) | |

TABLE 5
Gene expression levels for informative transcripts mapping to RIPK1, MLKL, and
RIPK3.

| Probe | Gene | Accession | Entrez | | | Average | | Adjusted |
|---|---|---|---|---|---|---|---|---|
| ID | Symbol | (GenBank) | ID | FC | t | Expression | P | P |
| 5560086 | RIPK1 | NM_003804 | 8737 | 0.222 | 6.890 | 7.351 | 7.5E−11 | 1.7E−09 |
| 4560541 | MLKL | NM_152649 | 197259 | 0.598 | 7.017 | 7.817 | 3.7E−11 | 9.5E−10 |
| 2710328 | MLKL | XM_001126647 | 197259 | 0.046 | 2.439 | 6.356 | 1.6E−02 | 2.6E−02 |
| 5810022 | MLKL | XM_001126647 | 197259 | 0.024 | 1.319 | 6.339 | 1.9E−01 | 2.4E−01 |
| 360142 | RIPK3 | NM_006871 | 11035 | 0.003 | −0.247 | 6.295 | 0.805 | 0.840 |

TABLE 6
Gene expression levels for the transcripts mapping to RIPK1, MLKL, and RIPK3
genes obtained from an independent dataset (GSE5281).

| Probe | Gene | | Entrez | | | Average | | Adjusted |
|---|---|---|---|---|---|---|---|---|
| ID | Symbol | Ens | ID | FC | t | Expression | P | P |
| 226551_at | RIPK1 | ENSG00000137275 | 8737 | 0.648 | 3.141 | 5.218 | 0.006 | 0.023 |
| 238025_at | MLKL | ENSG00000168404 | 197259 | 0.627 | 4.118 | 3.847 | 0.001 | 0.005 |
| 228139_at | RIPK3 | ENSG00000129465 | 11035 | 0.144 | 0.931 | 3.542 | 0.365 | 0.492 |

For Table 2: The groups were homogenous for age and gender distributions. Predictably, ApoE genotype and E4 carrier distributions were statistically different between CTL and AD. *t-test equal variance. **Fisher's exact test (20,000 simulated P values).

For Table 3: The groups were homogenous for height, weight, BMI, and expired age. Predictably, brain weight and MMSE were statistically lower in AD with respect to CTL. P (Sh): P values of Shapiro-Wilk normality test. P: P values for significance test between AD and CTL. For weight and MMSE Mann-Whitney U test was used. For all other variables t-test for equal variance was used. *MMSE: the bimodality of MMSE was assessed using visual inspection of density plot and Hartigan Test (Tot: P=0.0022; AD: P=0.0046; ND: P=2.2E-16).

For Table 4: Significance between AD and CTL was assessed using Fisher's Exact Test with 20,000 simulated P values.

For Table 5: Gene expression levels for all the informative transcripts mapping to RIPK1 (one probe), MLKL (three probes), and RIPK3 (one probe) in the Illumina HumanHT-12 v4 BeadChip. Fold change (FC) is expressed as Log2 fold change, so it is positive when the gene is overexpressed in AD and negative when the gene is underexpressed. The adjusted P values were corrected for 26,583 tests. RIPK1 mRNA levels were significantly higher in human AD brains. Out of the three probes mapping on MLKL, two resulted significantly different between CTL and AD. RIPK3 mRNA levels were not significantly different between the CTL and AD (n=97 for AD and n=98 for CTL).

For Table 6: Gene expression levels for the transcripts mapping to RIPK1, MLKL, and RIPK3 genes obtained from an independent dataset (GSE5281) characterized with Affymetrix Human Genome U133 Plus 2.0 Array. Microarray analyses were conducted after laser capture of neurons in the middle temporal gyms (n=12 for AD and n=6 for CTL). Fold change (FC) is expressed as Log2 fold change, so it is positive when the gene is overexpressed in AD and negative when the gene is underexpressed. The P values were corrected at the genome-wide level for 20,535 tests. RIPK1 and MLKL mRNA levels were significantly higher in human AD neurons. RIPK3 mRNA levels were not significantly different between the CTL and AD.

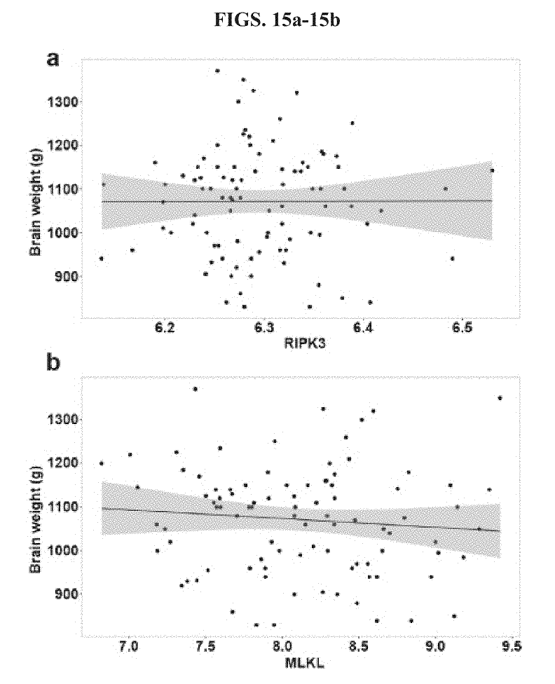

To probe for a linear relationship between gene expression and brain weight, we used a Pearson's correlation coefficient. While brain weight did not correlate with RIPK3 and MLKL levels (FIGS. 15a-15b and Table 7), we found a significant negative correlation between brain weight and RIPK1 expression in AD cases (FIG. 3d and Table 7). Given that brain weight changes in relation to height and gender, we performed a linear regression correcting for these covariates. The correlation was still significant (p=0.046), with a coefficient of β=−123.93 (Table 8). These data further corroborate that necroptosis may contribute to neuronal loss in sporadic AD.

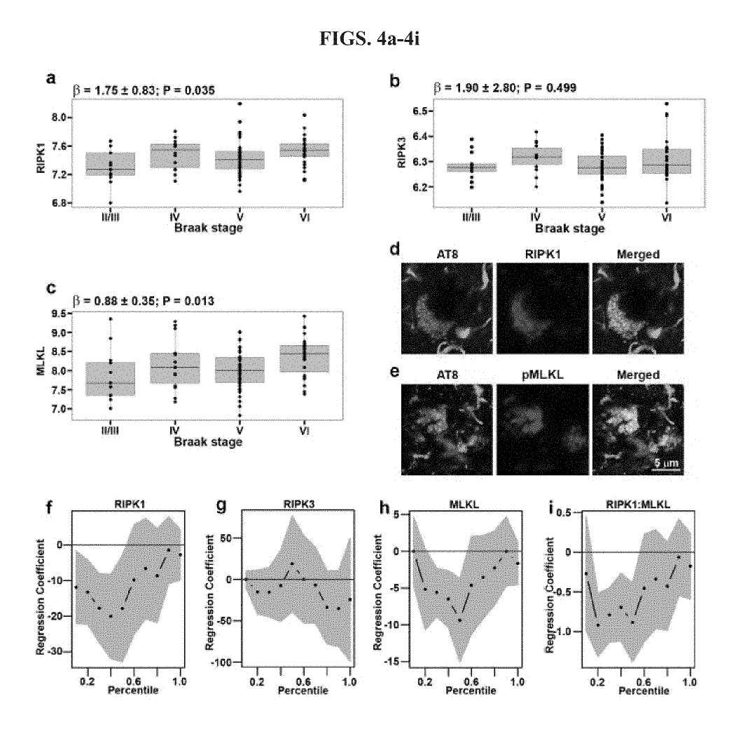

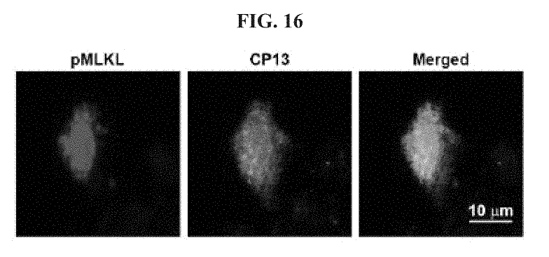

To determine the relationship between RIPK1, RIPK3, and MLKL levels and AD neuropathology, we performed an ordinal logistic regression with plaque density and Braak stage, which are two widely used approaches to evaluate the amount of neurofibrillary tangles and amyloid-βplaques, respectively^{6, 20}. None of the three necroptotic marker levels were predictive for the amyloid load (Table 8). In contrast, the increase of RIPK1 and MLKL mRNA levels, but not RIPK3, were significantly and positively correlated with the Braak stage (p<0.05; FIGS. 4a-4c and Table 8). This significance increased when we evaluated the effect of the interaction of RIPK1::MLKL (p=0.007; Table 8), which suggests that both markers covariate in the same samples together with the Braak stage. We also found that in CTL cases, there was a significant negative correlation between RIPK3 levels and Braak stage (p<0.001; Table 8). To further investigate the link between necroptosis and tau, we double stained sections from AD cases with AT8/RIPK1 and AT8/pMLKL. We found that 55.48±2.8% and 46.85±4.0% of the AT8 immunoreactivity co-localized with RIPK1 and pMLKL, respectively (FIGS. 4d-4e). We confirmed the colocalization between pMLKL and phosphorylated tau using CP13, which recognizes tau phosphorylated at Ser202, and an independent pMLKL antibody (FIG. 16). These data indicate a link between necroptosis and tau pathology but not between necroptosis and amyloid-β pathology. These findings are consistent with evidence indicating that tau pathology is more proximal than amyloid-β to neuronal loss in AD²¹.

TABLE 7
Pearson's correlation between brain weight and gene
expression levels of RIPK1, MLKL, and RIPK3.

| | | Pearson's Correlation | | | |
|---|---|---|---|---|---|
| | | AD | | CTL | |
| Gene | Clinical Variable | R | P | R | P |
| RIPK1 | Brain Weight | −0.333 | 1.1E−03 | 0.049 | 0.636 |
| MLKL | Brain Weight | −0.095 | 0.362 | −0.060 | 0.561 |
| RIPK3 | Brain Weight | 0.002 | 0.9858 | 0.188 | 0.066 |

TABLE 8
Linear (brain weight) and ordinal logistic (Braak stage and plaque density)
regressions using gene expression levels as predictors.

| | | Gene | Coeff | Std. Error | t | P |
|---|---|---|---|---|---|---|
| Brain | AD | RIPK1 | −123.93 | 60.65 | −2.043 | 4.6E−02 |
| Weight* | | MLKL | −0.780 | 21.1641 | −0.037 | 0.971 |
| | | RIPK3 | 5.91 | 180.41 | 0.033 | 0.974 |
| | ND | RIPK1 | 55.7 | 59.7 | 0.933 | 0.355 |
| | | MLKL | 2.35 | 19.04 | 0.123 | 0.902 |
| | | RIPK3 | 53.43 | 159.75 | 0.334 | 0.739 |
| Braak | AD | RIPK1 | 1.747 | 0.829 | 2.107 | 0.035 |
| Stage | | MLKL | 0.879 | 0.353 | 2.488 | 0.013 |
| | | RIPK3 | 1.901 | 2.809 | 0.677 | 0.499 |
| | | RIPK1:MLKL | 0.103 | 0.038 | 2.690 | 0.007 |
| | ND | RIPK1 | −0.037 | 0.895 | −0.041 | 0.967 |
| | | MLKL | 0.038 | 0.281 | 0.135 | 0.892 |
| | | RIPK3 | −8.105 | 2.424 | −3.344 | 8.2E−04 |
| | | RIPK1:MLKL | 0.002 | 0.031 | 0.076 | 0.940 |
| Plaque | AD | RIPK1 | 1.022 | 1.082 | 0.945 | 0.345 |
| Density | | MLKL | −0.202 | 0.426 | −0.474 | 0.636 |
| | | RIPK3 | −0.902 | 3.570 | −0.253 | 0.801 |
| | ND | RIPK1 | 0.972 | 0.905 | 1.074 | 0.283 |
| | | MLKL | 0.476 | 0.317 | 1.502 | 0.133 |
| | | RIPK3 | −2.585 | 2.260 | −1.144 | 0.253 |

For Table 7: Pearson's correlation between brain weight and gene expression levels of RIPK1, MLKL, and RIPK3. RIPK1 negatively correlates with brain weight in the AD group but not in the CTL group. MLKL and RIPK3 do not correlate with brain weight both in AD or CTL groups.

For Table 8: Brain weight regressions were corrected for height and gender (* in table). RIPK1 significantly correlates with brain weight in the AD group. No other correlations were detected either in the AD or CTL cases. RIPK1 and MLKL positively correlate with Braak stage only in the AD group. Notably, the interaction RIPK1::MLKL elicits a stronger correlation. These data suggest that RIPK1 and MLKL are coexpressed in the same samples, driven positive correlations with the Braak stage. RIPK3 negatively correlates with the Braak stage only in the CTL group. No correlations were detected for plaque density. SE: Standard Error; t: test statistic.

The Mini-Mental State Examination (MMSE) is a cognitive test routinely used to assess cognitive function²². To determine the relation between MMSE and necroptosis, we used a quantile regression due to the bimodal distribution of the MMSE scores. We ran 10 different models ranging from the 10th to the 100th percentiles for both CTL and AD. In CTL cases, RIPK1, RIPK3, and MLKL expression did not correlate with the MMSE (Table 9). In AD cases, for RIPK1, we obtained a negative significant regression (adj-p<0.05) for percentiles ranging from 30th (MMSE=5) to 40th (MMSE=7; Table 10; FIG. 4f). In contrast, RIPK3 did not correlate with the MMSE (FIG. 4g and Table 10). For MLKL, we obtained a negative significant regression (adj-p<0.05) in AD patients for percentiles ranging from the 30th (MMSE=5) to 50th (MMSE=12; FIG. 4h and Table 10). When we evaluated the RIPK1-MLKL interaction with MMSE, the regression was significant for a wider range of percentiles (20th-50th; FIG. 4i and Table 10), which suggests that RIPK1 and MLKL covariate in the same sample in relation to the MMSE.

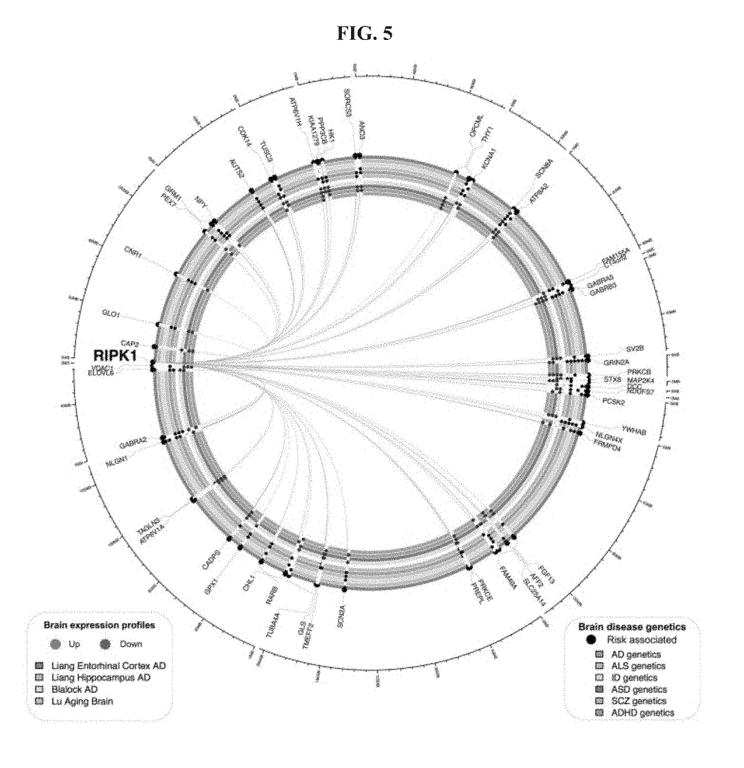

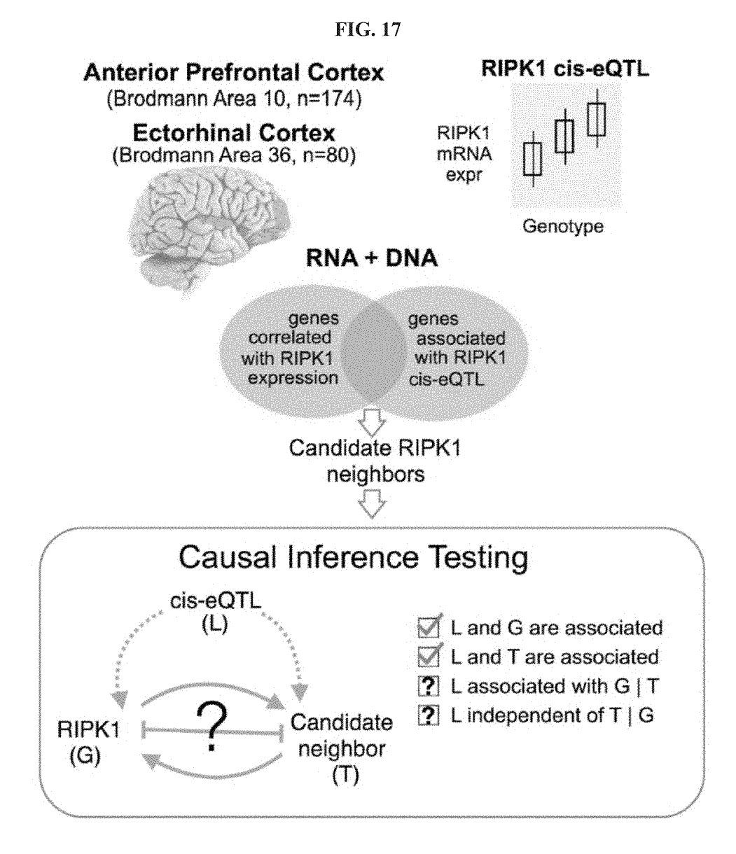

RIPK1 activity predicts gene expression dysregulation in AD: To better understand the regulatory context of RIPK1, we generated a causal gene regulatory network to model RIPK1 interactions in AD relevant tissue (FIG. 17). The network was inferred from DNA and transcriptomic data generated from post-mortem samples across two brain regions, which were used to build RIPK1 networks in the anterior prefrontal cortex (APFC, Brodmann Area 10, Total individuals: n=174, CTL: n=63, Definite AD: n=68, Likely AD: n=20, Possible AD: n=23) and the ectorhinal cortex (ERC, Brodmann Area 36, Total individuals: n=80, CTL: n=33, Definite AD: n=28, Likely AD: n=8, Possible AD: n=11) as represented in the Mount Sinai NIH Brain and Tissue Repository. This network inference approach leverages paired genetic and transcriptomic data, using cis-eQTL markers linked to RIPK1 as a causal anchor to resolve directed interactions between RIPK1 and its correlated genes through use of a causal inference test²³. This method of inference captures multi-order regulatory relationships that can include direct interactions between RIPK1 and its neighbors, as well as indirect effects that may be mediated through additional genes. Across both regions, we identified a total of 819 genes whose expression covariate with RIPK1 (APFC: 230 genes, 134 positively correlated, 127 negatively correlated; ERC: 598 genes, 183 positively correlated, 423 negatively correlated). Intriguingly, these genes significantly overlapped with multiple, independent AD differential gene expression profiles, comprising non-demented AD (which are characterized by moderate neuropathology, without cognitive impairment) and clinical AD²⁴, across multiple brain regions¹⁸ (FIG. 5, Table 11). There was uniform consistency between the signed relationship linking RIPK1 with its downstream neighbors, and the direction of differential expression in the clinical AD profiles; in particular, genes negatively regulated by RIPK1 overlapped substantially with genes down-regulated in AD. This large, concordant overlap between genes regulated by RIPK1, and genes differentially expressed across multiple AD severity and regional contexts, suggests that RIPK1 activity could explain a significant portion of described transcriptomic changes in AD. We also found that RIPK1 regulates multiple genes linked with risk associated variants for AD and other brain diseases (FIG. 5, Table 11).

To identify possible mechanisms underlying necroptosis activation in AD, we compared the set of genes regulated by RIPK1 with a set of genes associated with Z-VAD-FMK induced necroptosis in L929 cells²⁵. There was no significant overlap in either brain region (data not shown). In contrast, we found a strong overlap between genes regulated by RIPK1, and molecular functional enrichments for the mammalian target of rapamycin complex 1 (mTORC1) signaling (Table 11). These data are consistent with the reported role of mTOR and AKT1 in mediating neuronal necroptosis ²⁶. Taken together, these data indicate that in AD brains, necroptosis is activated by alternative pathways compared to those associated with Z-VAD-FMK induced necroptosis²⁵.

TABLE 9
Results for the quantile regression for ND samples.

| | | Percentile | | | | | | | | | |
|---|---|---|---|---|---|---|---|---|---|---|---|
| | | 10% | 20% | 30% | 40% | 50% | 60% | 70% | 80% | 90% | 100% |
| | | MMSE | | | | | | | | | |
| | | 26.0 | 26.0 | 27.0 | 28.0 | 29.0 | 29.0 | 29.0 | 30.0 | 30.0 | 30.0 |
| RIPK1 | Coeff | 1.208 | 0.000 | −1.686 | −2.710 | 0.000 | 0.000 | 0.000 | 0.000 | 0.000 | 0.000 |
| | SE | 2.110 | 2.481 | 3.166 | 3.093 | 2.413 | 2.191 | 1.910 | 1.294 | 0.408 | 0.000 |
| | t | 0.572 | 0.000 | −0.533 | −0.876 | 0.000 | 0.000 | 0.000 | 0.000 | 0.000 | −0.105 |
| | P | 0.570 | 1.000 | 0.597 | 0.385 | 1.000 | 1.000 | 1.000 | 1.000 | 1.000 | 0.917 |
| | Adj P | 1.000 | 1.000 | 1.000 | 1.000 | 1.000 | 1.000 | 1.000 | 1.000 | 1.000 | 1.000 |
| MLKL | Coeff | 0.320 | 0.000 | 0.000 | 0.869 | 0.813 | 0.584 | 0.543 | 0.000 | 0.000 | 0.000 |
| | SE | 0.699 | 0.863 | 0.964 | 0.826 | 0.600 | 0.414 | 0.327 | 0.375 | 0.105 | 1.625 |
| | t | 0.458 | 0.000 | 0.000 | 1.051 | 1.354 | 1.411 | 1.661 | 0.000 | 0.000 | 0.000 |
| | P | 0.649 | 1.000 | 1.000 | 0.298 | 0.182 | 0.165 | 0.103 | 1.000 | 1.000 | 1.000 |
| | Adj P | 1.000 | 1.000 | 1.000 | 0.746 | 0.606 | 0.606 | 0.606 | 1.000 | 1.000 | 1.000 |
| RIPK3 | Coeff | −5.848 | 0.000 | 0.000 | 5.181 | 0.000 | 0.000 | 0.000 | 0.000 | 0.000 | 0.000 |
| | SE | 3.936 | 4.906 | 5.166 | 4.912 | 4.680 | 2.964 | 2.980 | 2.400 | 1.018 | 0.000 |
| | t | −1.486 | 0.000 | 0.000 | 1.055 | 0.000 | 0.000 | 0.000 | 0.000 | 0.000 | 0.054 |
| | P | 0.144 | 1.000 | 1.000 | 0.297 | 1.000 | 1.000 | 1.000 | 1.000 | 1.000 | 0.957 |
| | Adj P | 1.000 | 1.000 | 1.000 | 1.000 | 1.000 | 1.000 | 1.000 | 1.000 | 1.000 | 1.000 |
| RIPK1:MLKL | Coeff | 0.039 | 0.000 | 0.000 | 0.000 | 0.089 | 0.067 | 0.069 | 0.000 | 0.000 | 0.000 |
| | SE | 0.079 | 0.098 | 0.112 | 0.103 | 0.084 | 0.057 | 0.053 | 0.037 | 0.021 | 0.155 |
| | t | 0.496 | 0.000 | 0.000 | 0.000 | 1.064 | 1.173 | 1.308 | 0.000 | 0.000 | 0.000 |
| | P | 0.622 | 1.000 | 1.000 | 1.000 | 0.292 | 0.247 | 0.197 | 1.000 | 1.000 | 1.000 |
| | Adj P | 1.000 | 1.000 | 1.000 | 1.000 | 0.975 | 0.975 | 0.975 | 1.000 | 1.000 | 1.000 |

TABLE 10
Results for the quantile regression for AD samples.

| | | Percentile | | | | | | | | | |
|---|---|---|---|---|---|---|---|---|---|---|---|
| | | 10% | 20% | 30% | 40% | 50% | 60% | 70% | 80% | 90% | 100% |
| | | MMSE | | | | | | | | | |
| | | 0.0 | 2.2 | 5.0 | 7.0 | 12.0 | 16.0 | 18.7 | 22.0 | 24.9 | 28.0 |
| RIPL1 | Coeff | −11.876 | −13.267 | −17.766 | −20.036 | −17.857 | −9.804 | −6.601 | −8.646 | −1.449 | −2.778 |
| | SE | 6.195 | 5.521 | 5.898 | 7.194 | 9.123 | 9.397 | 8.580 | 8.030 | 5.709 | 4.303 |
| | t | −1.917 | −2.403 | −3.012 | −2.785 | −1.957 | −1.043 | −0.769 | −1.077 | −0.254 | −0.646 |
| | R | 6.0E−02 | 1.9E−02 | 3.8E−03 | 7.1E−03 | 5.5E−02 | 3.0E−01 | 4.4E−01 | 2.9E−01 | 8.0E−01 | 5.2E−01 |
| | Adj P | 1.2E−01 | 6.5E−02 | 3.6E−02 | 3.6E−02 | 1.2E−01 | 4.3E−01 | 5.6E−01 | 4.3E−01 | 8.0E−01 | 5.8E−01 |
| MLKL | Coeff | 0.000 | −5.160 | −5.594 | −6.497 | −9.373 | −4.615 | −3.540 | −2.264 | 0.000 | −1.643 |
| | SE | 2.834 | 3.375 | 2.007 | 2.357 | 3.454 | 4.055 | 3.451 | 3.070 | 2.864 | 1.776 |
| | t | 0.000 | −1.529 | −2.788 | −2.756 | −2.713 | −1.138 | −1.026 | −0.737 | 0.000 | −0.925 |
| | P | 1.0E+00 | 1.3E−01 | 7.1E−03 | 7.7E−03 | 8.7E−03 | 2.6E−01 | 3.1E−01 | 4.6E−01 | 1.0E+00 | 3.6E−01 |
| | Adj P | 1.0E+00 | 3.3E−01 | 2.9E−02 | 2.9E−02 | 2.9E−02 | 5.1E−01 | 5.1E−01 | 5.8E−01 | 1.0E+00 | 5.1E−01 |
| RIPK3 | Coeff | 0.000 | 15.228 | 15.625 | −7.692 | 18.692 | 0.000 | −6.897 | 33.613 | −35.294 | −24.390 |
| | SE | 6.300 | 16.240 | 18.153 | 26.158 | 35.267 | 32.114 | 28.028 | 27.010 | 27.886 | 45.303 |
| | t | 0.000 | −0.938 | −0.861 | −0.294 | 0.530 | 0.000 | −0.246 | −1.244 | −1.266 | −0.538 |
| | P | 1.0E+00 | 3.5E−01 | 3.9E−01 | 7.7E−01 | 6.0E−01 | 1.0E+00 | 8.1E−01 | 2.2E−01 | 2.1E−01 | 5.9E−01 |
| | Adj P | 1.0E+00 | 9.8E−01 | 9.8E−01 | 1.0E+00 | 1.0E+00 | 1.0E+00 | 1.0E+00 | 9.8E−01 | 9.8E−01 | 1.0E+00 |
| RIPK1:MLKL | Coeff | −0.270 | −0.917 | −0.790 | −0.693 | −0.883 | −0.453 | −0.338 | −0.428 | −0.064 | −0.175 |
| | SE | 0.437 | 0.242 | 0.210 | 0.260 | 0.303 | 0.413 | 0.380 | 0.338 | 0.293 | 0.253 |
| | t | −0.618 | −3.784 | −3.768 | −2.663 | −2.911 | −1.096 | −0.889 | −1.267 | −0.217 | −0.690 |
| | P | 5.4E−01 | 3.6E−04 | 3.8E−04 | 9.9E−03 | 5.1E−03 | 2.8E−01 | 3.8E−01 | 2.1E−01 | 8.3E−01 | 4.9E−01 |
| | Adj P | 6.0E−01 | 1.9E−03 | 1.9E−03 | 2.5E−02 | 1.7E−02 | 4.6E−01 | 5.4E−01 | 4.2E−01 | 8.3E−01 | 6.0E−01 |

TABLE 11
Molecular functional enrichments of genes regulated by RIPK1.

| | | Genes | | |
|---|---|---|---|---|
| | | regulated by | Adj P | Fold |
| | Gene set | RIPK1 | value | change |
| AD differential | Liang AD Entorhinal Cortex - DOWN | negatively | 1.7E−04 | 1.31 |
| expression | Liang AD Hippocampus - DOWN | negatively | 5.6E−33 | 1.94 |
| | Liang AD Medial Temporal Gyrus - | negatively | 3.6E−57 | 2.20 |
| | DOWN | | | |
| | Liang AD Parietal Cortex - DOWN | negatively | 1.8E−44 | 2.14 |
| | Liang AD Parietal Cortex - UP | positively | 2.0E−03 | 1.45 |
| | Liang AD Superior Frontal Gyrus - | negatively | 8.8E−60 | 1.99 |
| | DOWN | | | |
| | Liang AD Visual Cortex - DOWN | negatively | 3.7E−26 | 2.15 |
| | Blalock AD Hippocampus - DOWN | negatively | 1.3E−29 | 3.03 |
| NDAD | Liang NDAD Entorhinal Cortex - UP | negatively | 1.6E−05 | 1.96 |
| differential | Liang NDAD Hippocampus - UP | negatively | 1.9E−03 | 1.39 |
| expression | Liang NDAD Medial Temporal Gyrus - | negatively | 1.1E−09 | 1.47 |
| | DOWN | | | |
| | Liang NDAD Medial Temporal Gyrus - | negatively | 4.5E−03 | 1.29 |
| | UP | | | |
| | Liang NDAD Visual Cortex - UP | negatively | 3.0E−02 | 1.43 |
| GWAS risk loci | Cognitive disorder\|DOID:1561 | negatively | 1.6E−02 | 1.20 |
| | Neurodegenerative disease\|DOID:1289 | negatively | 3.3E−02 | 1.26 |
| | Mood disorder\|DOID:3324 | negatively | 4.1E−02 | 1.26 |
| | Synucleinopathy\|DOID:0050890 | negatively | 4.7E−02 | 1.51 |
| | Parkinson's disease\|DOID:14330 | negatively | 4.7E−02 | 1.51 |
| | Demential DOID:1307 | negatively | 5.2E−02 | 1.32 |
| | Alzheimer's disease\|DOID:10652 | negatively | 5.7E−02 | 1.32 |
| | Tauopathy\|DOID:680 | negatively | 5.7E−02 | 1.32 |
| | Disease of mental health\|DOID:150 | negatively | 6.3E−02 | 1.14 |
| | Central nervous system disease\|DOID:331 | negatively | 6.3E−02 | 1.21 |
| | Sensory system disease\|DOID:0050155 | negatively | 6.6E−02 | 1.35 |
| Age Related | LU AGING BRAIN - DOWN | negatively | 3.5E−09 | 4.58 |
| mTOR and AKT | CREIGHTON - AKT1 signaling via | negatively | 1.4E−02 | 5.93 |
| signaling | mTOR DN | | | |
| | AKT UP mTOR DN.V1 UP | positively | 6.2E−02 | 2.63 |
| | AKT UP.V1 UP | positively | 7.6E−02 | 2.52 |
| | AKT1 protein complex: a-AKT | negatively | 2.6E−04 | 3.18 |
| | AKT1S1 protein complex: BL4782 | negatively | 1.7E−02 | 2.03 |
| | Hallmark mTORC1 signaling | negatively | 4.0E−05 | 3.01 |
| | mTOR protein complex: BL4773 | negatively | 9.3E−02 | 2.15 |

For Table 9: Results for the quantile regression in the ND samples for RIPK1, MLKL, RIPK3, and for the interaction between RIPK1 and MLKL for percentiles from 10^{th} to 100^{th} (10% increase). No significance was detected in any case. SE =Standard Error; t=test statistic.

For Table 10: Results for the quantile regression in the AD samples for RIPK1, MLKL, RIPK3, and for the interaction between RIPK1 and MLKL for percentiles from 10^{th} to 100^{th} (10% increase). RIPK1 and MLKL negatively correlate with MMSE for a wide range of percentiles (P value and adjusted P value<0.05 in red in the table). Notably, the interaction RIPK1::MLKL elicits a stronger and wider correlation, suggesting that RIPK1 and MLKL are coexpressed. SE=Standard Error; t=test statistic.

For Table 11: We identified significant enrichments (FDR<0.1) between genes regulated by RIPK1 (in APFC or ERC) and multiregional brain transcriptome profiles for AD, NDAD, neurodegenerative disease GWAS risk loci sets and mTOR signaling pathways (NDAD: Non-Demented Alzheimer's Disease).

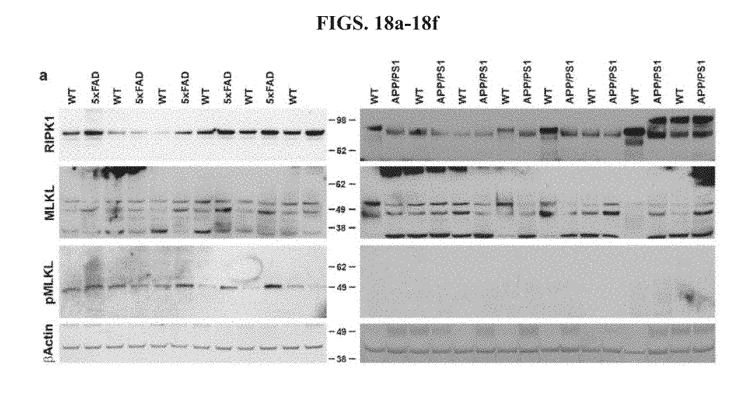

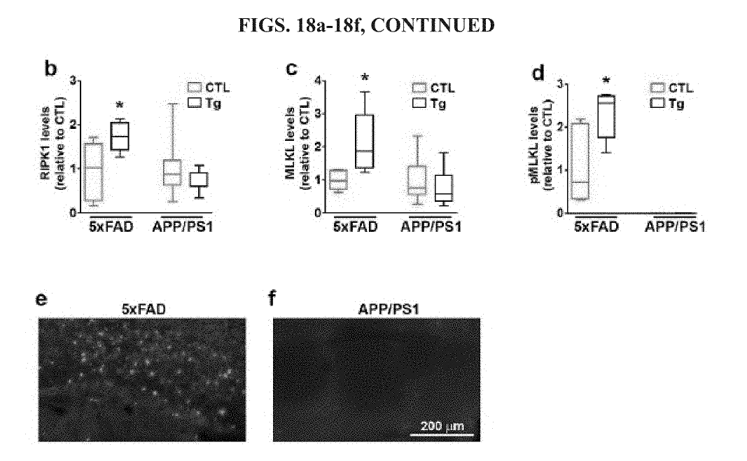

Inhibiting necroptosis reduces neuronal death in 5xFAD mice: To further probe for a role of necroptosis in AD, we first measured necroptotic markers in two widely used animal models of AD, known as 5xFAD and APP/PS1 mice^{27, 28}. We found that the levels of RIPK1, MLKL, and pMLKL were not significantly different between 12-month-old APP/PS1 and non-transgenic littermates (FIGS. 18a-18d). In contrast, RIPK1, MLKL, and pMLKL levels were significantly higher in the brains of 11-month-old 5xFAD mice compared to non-transgenic littermates (FIG. 18a-18d). Therefore, in 5xFAD mice, which are characterized by marked cell loss (FIG. 18e and ref. 27), necroptotic markers were significantly increased. In contrast, in APP/PS1 mice, which do not demonstrate overt cell loss (FIG. 18f and ref. 28) necroptotic markers were not statistically different from non-transgenic mice.

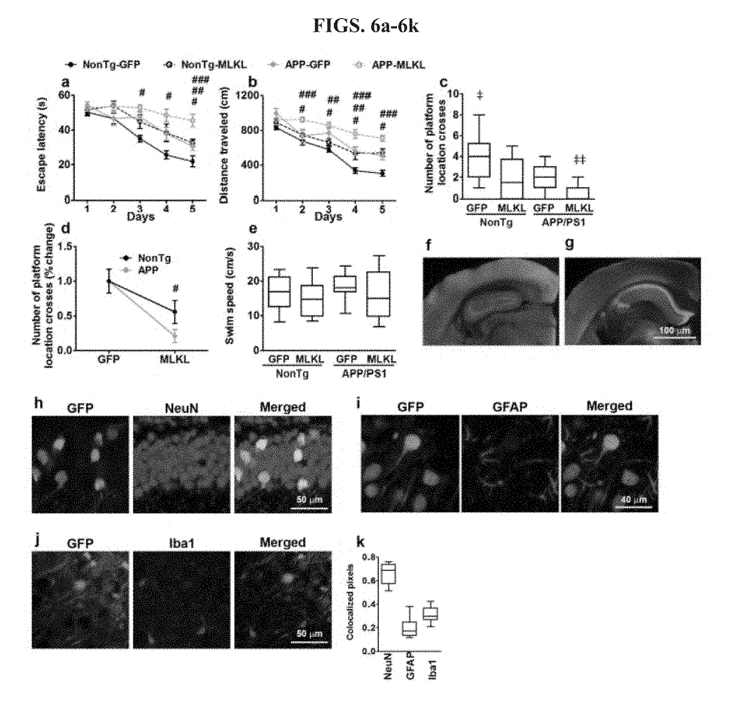

To further study the role of necroptosis in AD neuropathology, we sought to determine the impact of inducing necroptosis on AD-like pathology in mice. To this end, we generated an adeno-associated virus (AAV) expressing a constitutively active form of MLKL (caMLKL, which has been previously described²⁹), under a neuronal specific promoter. To allow co-expression of the green fluorescent protein (GFP) with caMLKL, we cloned a 2A-like peptide sequence between the caMLKL cDNA and the GFP cDNA. The virus was stereotaxically injected into the third ventricle of 3-month-old APP/PS1 mice (n=14 mice, 6 females and 8 males) and non-transgenic (NonTg) littermates (n=12 mice, 5 males and 7 females), using the following coordinates from Bregma: AP −2.46 mm; DL 0 mm; DV -2 mm. Control age- and gender-matched APP/PS1 and NonTg mice (n=14 mice/genotype) were injected with AAVs expressing GFP alone. Three months after the surgeries, mice were tested on the spatial version of the Morris water maze (MWM). We trained mice four times per day for five consecutive days to learn the location of a hidden platform using cues located outside the maze. Using a two-way ANOVA, we found that for the escape latency there was a significant effect for days (F=31.26; p<0.0001) and genotype (F=21.37; p<0.0001) as well as a significant genotype x day interaction (F=2.16; p<0.05; FIG. 6a). The effect of day indicated that all mice learned the task across days whereas the effect of genotype indicated that one or more genotypes had a different pace of learning from each other. A post hoc test with Bonferroni's correction showed that APP/PS1 and NonTg mice injected with the caMLKL virus (APP/PS1-MLKL; NonTg-MLKL) performed significantly worse than the genotype-matched mice injected with the GFP virus (APP/PS1-GFP) and (NonTg-GFP) at day 4 and day 5, respectively. Notably, the APP/PS1-MLKL mice performed significantly worse than NonTg-MLKL mice at Day 5. When we analyzed the distance traveled to find the platform, we found a significant effect for days (F=45.48; p<0.0001) and genotype (F=31.46; p<0.0001), as well as a significant genotype x day interaction (F=1.872; p<0.05; FIG. 6b). A post hoc test with Bonferroni's correction showed that the NonTg-GFP performed significantly better than any other group at days 4 and 5. Notably, the APP/PS1-MLKL performed significantly worse than APP/PS1-GFP at days 2, 4, and 5; and significantly worse than NonTg-MLKL at days 3 and 4.

Twenty-four hours after the last training trial, we conducted probe trials to evaluate spatial reference memory. Specifically, we measured the number of platform location crosses and the swim speed during a single 60-second trial. One-way ANOVA indicated a significant difference among the four groups (F=11.59; p<0.0001; FIG. 6c). Bonferroni's post hoc analyses indicated that the APP/PS1-MLKL performed significantly worse than the other three groups. To assess the degree of exacerbation induced by increasing MLKL levels, we normalized the number of platform location crosses for the two genotypes in relation to mice that received the GFP virus. We found a steeper slope for the APP/PS1 mice compared to the NonTg mice, indicating that the MLKL virus had more severe effects on spatial memory in transgenic mice than in NonTg mice (FIG. 6d). We also measured the swim speed of mice during the probe trials and found that it was not statistically significant among the four genotypes (FIG. 6e), indicating that the effects on learning and memory were independent of physical performance. Taken together, these findings indicate that inducing necroptosis exacerbates cognitive decline in APP/PS1 mice to a greater degree than NonTg mice.

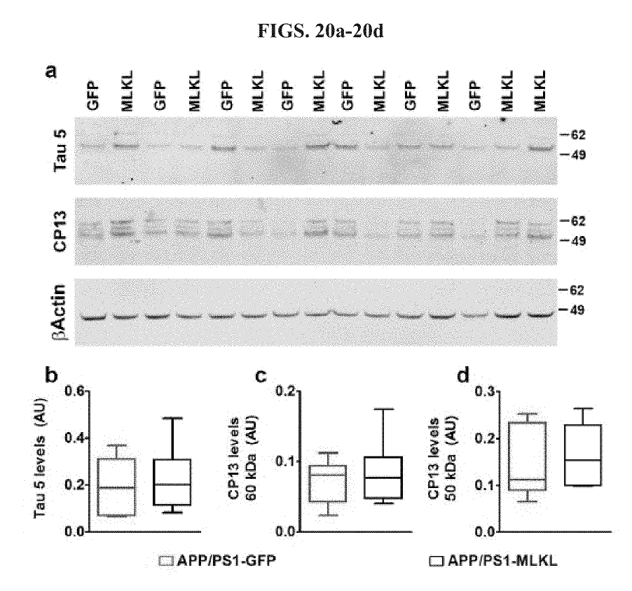

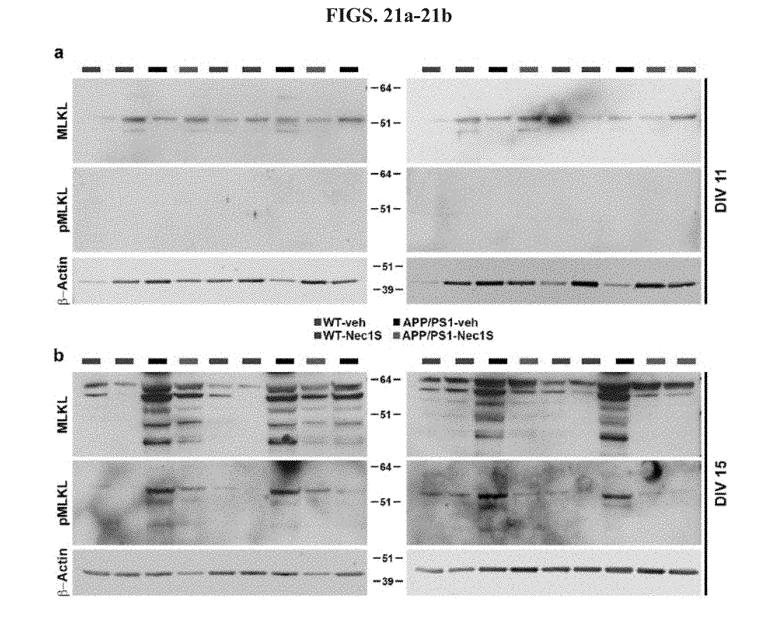

At the end of the behavioral tests, we assessed the extent of viral diffusion, as well as the specific cell type infected by the virus. To this end, we stained sections from APP/PS1-GFP and NonTg-GFP mice with an anti-GFP antibody. Consistent with the injection site, we observed strong GFP immunoreactivity in the hippocampus and adjacent cortical areas (FIGS. 6f-6g). Confocal imaging of sections from APP/PS1-GFP mice indicated a 66.84±3.2% co-localization between GFP and the neuronal maker NeuN (FIGS. 6h, 6k), 20.06±3.0% co-localization between GFP and the astrocyte marker GFAP (FIGS. 6i, 6k), and 31.14±2.4% co-localized between GFP and the microglia marker Ibal (FIGS. 6j-6k). One way ANOVA indicated that these values were significantly different among each other (F=70.35; p<0.001). Bonferroni's multiple comparison test indicated that all three groups were statistically significant from each other. Consistently with the AAV promoter used, these data indicate that caMLKL is preferentially expressed in neurons.

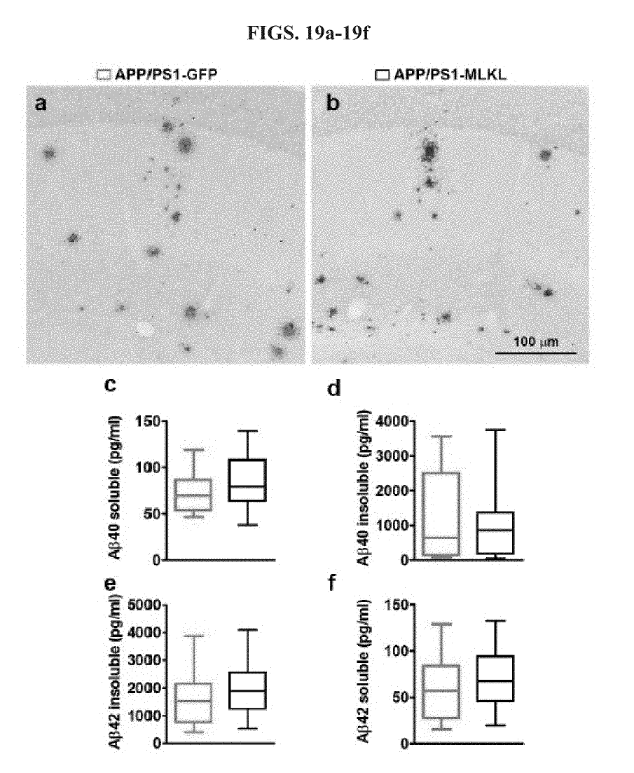

To probe for the mechanisms underlying the caMLKL-mediated changes in cognitive function, we assessed AP pathology, endogenous tau levels, and neurodegeneration. We stained hippocampal sections from APP/PS1-GFP and APP/PS1-MLKL mice with an Aβ42-specific antibody and found that AP load was similar between the two groups (FIGS. 19a-19b). To better quantify changes in AP, we measured soluble and insoluble Aβ40 and Aβ42 by sandwich ELISA. Consistent with the immunohistochemical data, we found no changes in Aβ levels between the two groups for any of the measurements (FIGS. 19c-19f). We also measured endogenous tau levels by western blot. The levels of total tau, measured by the tau-5 antibody, were similar between the two groups (FIGS. 20a-20b). Similarly, the levels of tau phosphorylated at Ser202 (as detected by the CP13 antibody) were not statistically different between APP/PS1-GFP and APP/PS1-MLKL mice (FIGS. 20a, 20c-20d). The lack of changes in Aβ and tau, indicate that under these conditions, inducing necroptosis does not contribute to Aβ and tau accumulation.

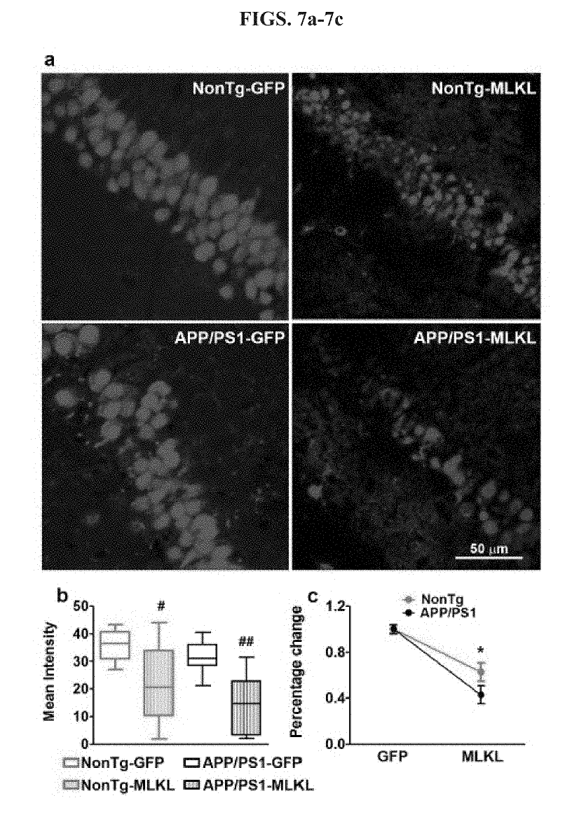

We then assessed the degree of neuronal loss by staining hippocampal sections with NeuN, a commonly used neuronal marker (FIG. 7a). Quantitative analyses of the NeuN staining indicated a significant reduction in the number of neurons among the four groups (F=21.77; p<0.0001; FIG. 7b). Bonferroni's post hoc analyses indicated that the mice injected with the caMLKL virus had significantly fewer CA1 neurons compared to genotype-matched mice injected with the GFP virus (FIG. 7b). Most notably, the number of NeuN-positive neurons in the APP/PS1-MLKL group was significantly lower compared to the number of NeuN-positive neurons in the NonTg-MLKL group. This difference is further highlighted when we analyzed the data as a percentage change in neuronal loss between the GFP- and the MLKL-injected mice. These data indicate that the degree of MLKL-induced cell loss is higher in APP/PS1 mice than in NonTg mice (FIG. 7c), suggesting that the APP/PS1 brains are more susceptible to activation of necroptosis.

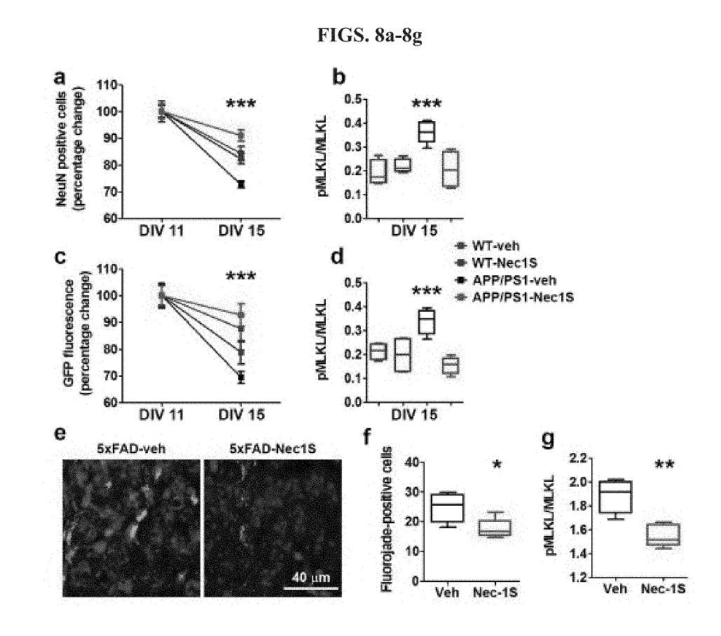

To probe for a direct link between necroptosis and neuronal loss in AD, we plated primary cortical neurons from APP/PS1 and wild type littermate mice. After seven days in vitro (DIV), neurons were treated with 10 μM of the necroptosis inhibitor 7-Cl-O-necrostatin (Nec15) or vehicle. Treatment was repeated at DIV 11. We then fixed neurons at DIV 11 and 15, and stained them for NeuN to count total number of neurons. Using a two-way ANOVA, we found that there was a significant effect for time (F=76.13; p<0.0001) and groups (F=7.109; p=0.0002) as well as a significant time x group interaction (F=7.109; p=0.0002; FIG. 8a). A post hoc test with Bonferroni's correction showed that the number of neurons were similar among the four groups at DIV 11. In contrast, at DIV 15, the total number of neurons in the APP/PS1-veh group was significantly different compared to the other three groups. Notably, the APP/PS1-Nec1S group had more neurons than the APP/PS1-veh group, and as many neurons as both WT groups. Notably, Nec1S did not significantly affect the decrease in the number of wild type neurons. To determine changes in necroptosis, we isolated proteins from neurons of the same four groups and measured the levels of total and phosphorylated MLKL by western blot. We found that pMLKL was not detectable at DIV 11 in all four groups (FIG. 2l). In contrast, we found that the ratio pMLKL/MLKL was significantly at DIV 15 (F=10.20; p=0.0008). This difference was driven by the APP/PS1-veh group, which ratio pMLKL/MLKL was statistically different compared to the other three groups (FIG. 8c and FIG. 2l).

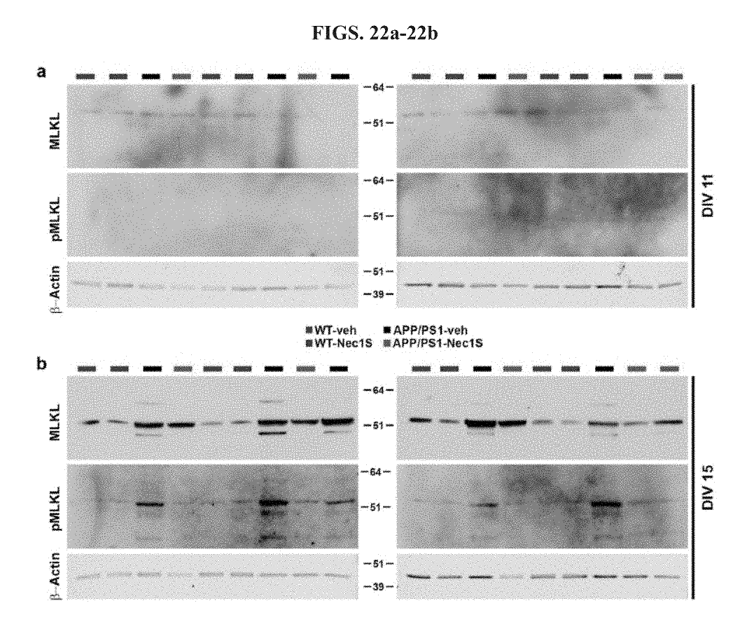

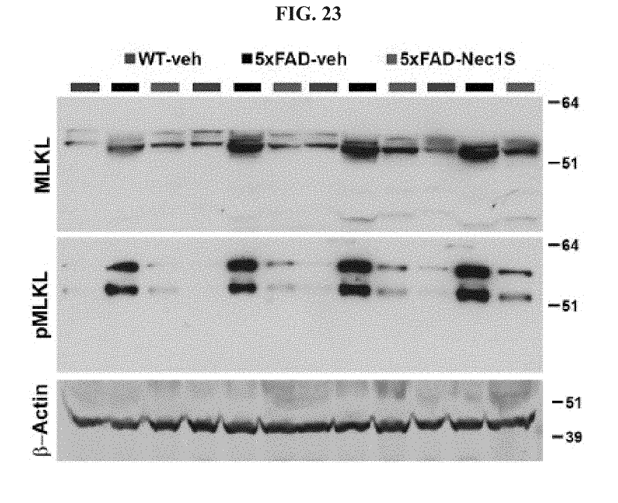

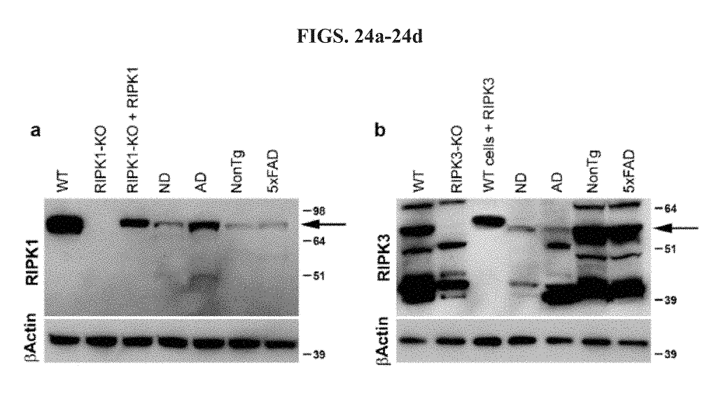

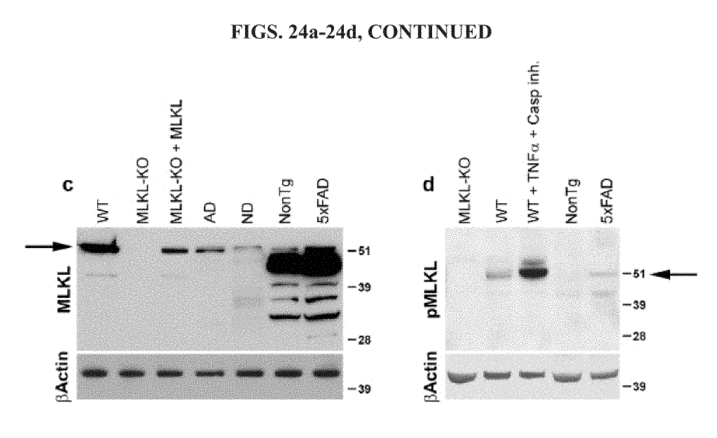

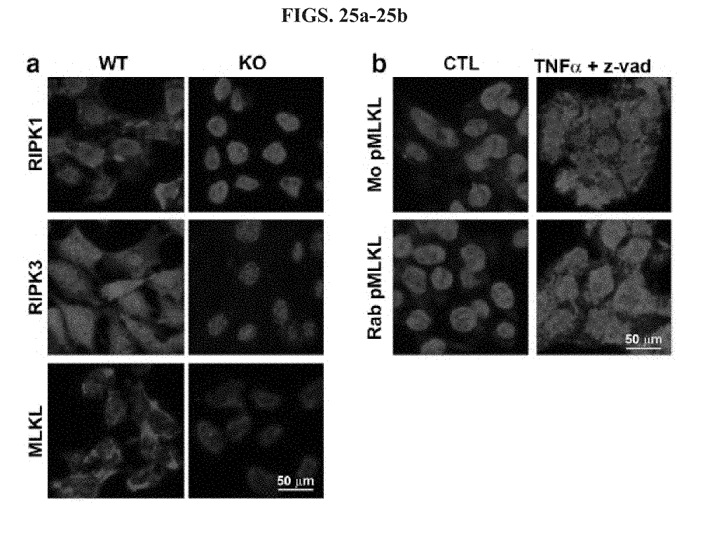

To validate these results, we repeated the experiments under different experimental conditions. At DIV 1, we infected primary cortical neurons isolated from APP/PS1 and wild type mice with an adeno-associated virus (AAV) expressing the green fluorescent protein (GFP) under the neuronal promotor CamKII. At DIV 7 and 11, neurons from the two genotypes were treated with 10 μM Nec1S or vehicle. We then measured GFP fluorescence in live cells as an indication of live neurons. Using a two-way ANOVA, we found that there was a significant effect for time (F=170.2; p<0.0001) and groups (F=102.4; p<0.0001) as well as a significant time x group interaction (F=78.43; p<0.0001; FIG. 8d). A post hoc test with Bonferroni's correction showed that the GFP fluorescence was similar among the four groups at DIV 11. In contrast, at DIV 15, the APP/PS1-veh group had lower GFP levels compared to the other three groups. Consistent with the previous experiments, pMLKL levels were not detectable at DIV 11 in any of the four groups (Supplementary FIG. 14). In contrast, pMLKL levels were significantly different among the four groups at DIV 15 (F=11.30; p<0.0005). This difference was driven by the APP/PS1-veh group, which ratio pMLKL/MLKL was statistically different compared to the other three groups (FIG. 8f and FIG. 22). Taken together, the results of these two independent experiments indicate that Nec-1S reduces the accelerated in vitro cell death of APP/PS1 primary cortical neurons.

To validate these data in vivo, we treated 8-month-old 5xFAD mice with Nec-1S (n=5) or vehicle (n=4). Specifically, mice receive a single intraperitoneal injection of 10 mg/kg Nec1s after which they were given Nec1S in their drinking water at a concentration of 0.5 mg/ml for 21 days. We adjusted the dosing regimen from a previous report that showed feasibility of the treatment and brain bioavailability of the drug'. Notably, Nec1S-treated 5xFAD mice had fewer Fluoro-Jade-positive neurons compared to vehicle-treated 5xFAD mice (t=2.627; p=0.0341; FIG. 8g-h, and FIG. 23). Western blot data confirmed that necroptosis was reduced in Nec1S-treated 5xFAD mice, as indicated by a lower pMLKL/MLKL ratio (t=4.35; p=0.0033; FIG. 8i and FIG. 23). Overall, these data provide compelling evidence that necroptosis contributes to neuronal loss in 5xFAD mice.

Discussion

Neuronal loss is a cardinal feature of AD and invariably affects multiple brain regions. Notably, brain atrophy is evident in asymptomatic individuals 10 years before the onset of dementia³⁰. Despite this indisputable evidence, the precise mechanism by which neurons die is still unknown³¹⁻³³. Identifying the mechanisms leading to neuronal loss in AD is fundamental for the development of an efficient therapeutic strategy to treat or slow down the progression of AD. Instead, the vast majority of ongoing clinical trials are designed to reduce a toxic insult (e.g., removal of AP from the brain). However, given that the exact trigger of AD is unknown, and given the growing appreciation for the fact that multiple causes of the disease are extremely likely, targeting the mechanisms of neurodegeneration is critical as it may have beneficial effects independently of the trigger³⁴. Alternatively, it could be used in concomitance with other therapeutics that aim at blocking the neurotoxic insult. Here, we report novel and exciting data expected to fill this critical gap in knowledge; we provide the first direct evidence that necroptosis is activated in human AD brains, as well as in a mouse model of AD that develops neuronal loss. Our studies open new venues of research and interventions for this insidious disorder, which affects more than 40 million people worldwide¹. From a basic biology prospective, it will be fundamental to dissect the mechanisms underlying necroptosis induction in AD; such studies may uncover new and critical knowledge into the pathogenesis of this disorder. From a therapeutic prospective, these data strongly suggest that reducing necroptosis may be a valid therapeutic target for AD.

There is a large body of evidence suggesting that within AD brains there is a favorable environment for apoptosis. For example, DNA fragmentation has been detected in postmortem human brains³⁵. Additionally, activation of several caspases has also been reported. However, it needs to be noted that caspase activation and DNA fragmentation do not necessarily lead to apoptosis³⁶. For example, there is a large body of literature indicating that caspase-3 activation might be involved in synaptic function in the adult brain³⁷; while DNA fragmentation routinely occurs during DNA repair in response to various stressors³⁸. Apoptotic bodies and/or chromatin condensation, which are more proximal to death by apoptosis, have not been reported in AD³⁹. It is estimated that it takes up to 24 hours for a neuron to undergo apoptosis. Thus, given the extensive number of neurons reported with DNA fragmentation and other apoptotic features, if apoptosis were the only key factor involved in neuronal loss in AD, this disorder would be an acute one, instead of the chronic, slow progressive disease that it is³³. It is tempting to speculate that early activation of caspases and DNA fragmentation, in the absence of apoptotic bodies and chromatin condensation, might instead be evidence of necroptosis activation. Indeed, recent evidence highlighted an extensive crosstalk between apoptosis and necroptosis⁴⁰.

Necroptosis contributes to the pathogenesis of several neurodegenerative disorders, such as amyotrophic lateral sclerosis (ALS) and multiple sclerosis (MS)^{13, 14}. The three key molecular mediators of necroptosis are RIPK1, RIPK3, and MLKL. Our data consistently show, across different cohorts, that RIPK1 levels are increased in human AD brains. While the mechanisms regulating RIPK1 expression in AD remain elusive, it is known that the activation of this kinase can be regulated by different signaling pathways including chronic TNFa-mediated inflammation, which is a feature of AD⁴¹. Once activated, RIPK1 protects cells from caspase-8 mediated apoptosis^{42, 43}. In contrast, chronic activation of RIPK1 leads to necroptosis activation. Most notably, we generated a causal gene regulatory network to model RIPK1 regulatory activity in AD relevant tissue. Our data clearly indicate that upregulation of RIPK1 alone may account for the gene expression changes independently reported in several gene expression experiments using AD brains. These findings are consistent with previous reports indicating that RIPK1 is involved in the formation and regulation of protein complexes involved in gene regulation⁴⁰.

Although RIPK3 expression levels were not statistically different between AD and CTL cases, we reported that RIPK3 is associated with RIPK1 and MLKL to a greater degree in AD brains compared to CTL cases. These findings are consistent with the overall mechanisms underlying necroptosis activation: RIPK1 binds to RIPK3, which in turn binds to MLKL to form the necrosome. In other words, necrosome formation, which is an invariable marker of necroptosis activation, is dependent on the physical interaction between RIPK1, RIPK3, and MLKL, and not on the relative levels of RIPK3. After the necrosome forms, RIPK3 phosphorylates MLKL, which becomes the executioner of necroptosis. Once phosphorylated, MLKL aggregates to form homodimers, binds to phosphatidylinositol phosphates, permeabilizes membranes, and induces cell death^{9, 10}. Notably, phosphorylation and dimerization of MLKL are sufficient and necessary for necroptosis induction⁴⁴⁻⁴⁶. Consistent with these data, we report that MLKL phosphorylation and aggregation is increased in AD brains, driving its localization to the membrane. Moreover, we report that RIPK1 and MLKL correlate with Braak stage only in AD patients. These data suggest that tau accumulation could be a key trigger in necroptosis activation; consistently it has been shown that NFTs represent a proximal event to neuronal loss in AD and correlate well with cognitive function⁴⁷. To this end, our data show that for both Braak stage and MMSE, the significance of the correlation increases when we evaluate the interaction of RIPK1-MLKL, leading to the hypothesis that it is not only the absolute expression of these proteins that may contribute to cell loss in AD, but also their co-expression.

In summary, this Example demonstrates the first direct evidence of necroptosis activation in AD. These findings may serve as a spring board for future in depth evaluation of the triggers of necroptosis in AD. Moreover, these data support the use of a therapeutically effective amount of a compound that inhibits receptor-interactive protein kinase 1 (RIPK1) or Mixed Lineage Kinase Domain-like (MLKL), or a pharmaceutically acceptable salt thereof, in a dosage sufficient to decrease necroptosis.

Materials and Methods

Human tissue and mice: The human samples were obtained from the Brain and Body Donation Program at the Banner Sun Health Research Institute, whose average PMI is 2.75 hours⁴⁸. The cases were selected randomly by personnel of the Brain and Body Donation program among the tissue available. Groups were matched based on their clinical and neuropathological phenotype. The generation of APP/PS1 and 5xFAD has been described previously^{27, 28}. We backcrossed the APP/PS1 to a pure 129S6 background for 12 generations. The 5xFAD mice are on a C57B1/6 background. All mice were housed 4-5 per cage at 23° C., kept on a 12 h light/dark cycle, and were given ad libitum access to food and water. Mice were randomly assigned to a specific group based on their genotype and there were no other factors that determined groups' selection. No mice were excluded from the statistical analyses. For the behavioral analyses (see below), the experimenters were blinded to genotype and treatment. No blinding was done for the other experiments. All animal procedures were approved by the Institutional Animal Care and Use Committee of the Arizona State University.

Immunohistochemistry: Coronal sections from hippocampus and temporal gyms of control (CTL) and Alzheimer's disease (AD) cases were pre-mounted on coated slides and washed in TBS. After blocking in TBSB (TBS, 2% BSA, and 0.1% Triton X), tissue was incubated overnight at 4° C. with the following primary antibodies: RIPK1 (1:200, BD Transduction Laboratories, catalog number 610459), MLKL (1:200, Novus Biologicals, catalog number NBP1-56729), RIPK3 (1:200, RD Systems catalog number MAB7604), pMLKL Ser358/Thr357 (1:200, Abcam, catalog number ab187091), pMLKL (1:200, SAB Signalway Antibody, catalog number 12837), NeuN (1:200, Cell Signaling, catalog number 024307P), Ibal (1:200, Wako, catalog number 09-19741), GFAP (1:200, Cell Signaling, catalog number 3670S), Caspase 3 (1:200, Millipore, catalog number AB3623), phospho-tau pSer202/Thr205 (ATB, 1:200, Thermo Fisher Scientific, catalog number MN1020), and pan-Cadherin (1:200, Abcam, catalog number ab6528 and ab6529). CP13 was a kind gift from Dr. Peter Davies. Sections were washed to remove the excess of primary antibody and incubated in the appropriate secondary fluorescent antibodies for 1 hour at RT (1:200, Thermo Fisher Scientific, catalog numbers A-11034, A-11034, A21429, and A-21424). The excess of secondary antibody was washed and the sections were coverslipped with Prolong® Diamond® antifade mountant (Thermo Fisher Scientific). For Fluoro-Jade staining, tissue from APP/PS1 and 5xFAD mice was mounted on coated slides and rehydrated on decreasing concentrations of ethanol. After rehydration the tissue was pretreated for 5 min in potassium permanganate and then incubated for 30 min in the dark at room temperature in Fluoro-Jade (Millipore) and DAPI (Sigma-Aldrich). The tissue was then washed in water and let dry overnight. The next day the slides were cleared in xylene and coverslipped with DPX mounting solution (Sigma-Aldrich). The immunohistochemistry on the brain sections from APP/PS1 was conducted following a similar protocol as we previously detailed 49. In this case, sections were stained with an anti-A(342 specific antibody (1:200; Millipore, catalog number AB5078P) and with a neuronal marker NeuN (1:200, Millipore, catalog number MAB377), Iba1 (Wako), GFAP (Cell Signaling) and GFP (1:200, Thermo Fisher Scientific, catalog number A-11122).

Confocal microscopy and image analysis: All images were acquired at a resolution size of x=1024, y=1024, and z=1, using a 63× oil immersion objective and zoom of 1.5 using the Leica DM 2500 confocal microscope. Image fields within the x-y plane of the tissue section were randomly selected. For each image acquired, channels were split to allow separate pixel quantification in both the 561 nm and 635 nm channels. To quantify total protein in each image field, channels were split and the integrated signal density of each channel was quantified using ImageJ software. Human brain tissue sections immunostained for RIPK1, RIPK3, MLKL, and pMLKL were analyzed to measure absolute protein levels, as well as co-localization between RIPK1 and RIPK3, RIPK3 and MLKL, and RIPK1 and MLKL. Human brain tissue sections were also immunostained with both the necroptosis marker, pMLKL, as well as one of three cell type markers, NeuN, GFAP, or Iba1. These experiments were used to measure co-localization of pMLKL with these three cell specificity markers. The same process was also carried out with human brain sections immunostained with RIPK1 and pTau, as well as pMLKL and pTau (both AT8 and CP13). To measure the proportion of pMLKL localized to the plasma membrane in human AD brains, brain tissue sections were immunostained with pMLKL and pan-cadherin antibodies. For all co-localization measurements in human tissue, lasers 561 nm and 635 nm were used for excitation of secondary antibody fluorophores Alexa555 and Alexa647, respectively. To obtain a Pearson Correlation Coefficient (PCC), ImageJ plugin ‘Coloc2’ was used. This plugin was also used to obtain a Costes randomization p-value (using 1000 Costes randomizations), in order to assess significance of obtained PCC values compared to PCC values obtained by random shuffling of the two channels. The Coloc2 plugin was also used to obtain the Mander's Correlation values for intensities above Costes threshold (tml and tm2), which were used to obtain the ratio of pixels from one channel that co-localize with total pixels in the second channel (i.e., ratio of pMLKL pixels that co-localize with total AT8 pixels). The ratio of co-localized pixels were similarly obtained in brain sections from mice injected with AAV-GFP-MLKL or AAV-GFP, to determine the cell type in which the viruses were selectively expressed.

Protein Extraction: Human temporal gyms tissue samples (0.1 g/sample) were extracted in sequential buffers, from lower to higher detergent strength. Briefly, the samples were homogenized in TBS (50 mM Tris-HCl, pH 7.4, 175 mM NaCl, 5 mM EDTA, 1 mM N-ethyl-maleimide) containing protease inhibitors (Roche) and phosphatase inhibitors (Millipore). The protein extract was centrifuged at 120,000 g for 30 minutes at 4° C. The resulting supernatant represented the TBS soluble fraction. Pellets were homogenized in TBS/1% Triton X-100 and spun at 1000 g for 10 minutes at 4° C. The supernatant represented the Triton soluble fraction. Pellets were washed in TBS/1M sucrose and then homogenized in RIPA buffer. The samples were centrifuged at 1000 g for 10 minutes at 4° C. and supernatant was collected and stored as RIPA fraction. The detergent-insoluble pellets were then re-suspended in 8 M urea/5% SDS and stored as urea fraction. The frozen mouse hemispheres were homogenized in 1 ml of cold T-PER (Thermo Fisher Scientific) protein extraction buffer containing complete protease inhibitor (Roche) and phosphatase inhibitor (Millipore). Brain homogenates were ultra-centrifuged at 100,000 g for 1 h at 4° C. The supernatant was recovered and stored at −80° C. until used for western blots.

Western blot and ELISA: The proteins were run using reducing conditions in precast gels (Thermo Fisher Scientific) and transferred to nitrocellulose membranes (iBlot, Thermo Fisher Scientific). The membranes were incubated for 60 min in 5% nonfat milk (Great Value) in Tris-buffered saline with Tween (TBST; 0.1 M Tris, 0.15 M NaCl, and 0.1% Tween 20). After blocking, membranes were incubated overnight at 4° C. in 5% milk in TBS-T with the appropriate primary antibodies: RIPK1 (1:1000, BD Biosciences, catalog number 610459), RIPK3 (1:1000, Novus, catalog number NBP1-77299), MLKL (1:1000, Novus, catalog number NBP1-56729), pMLKL (1:000, Abcam, catalog number ab196436), tau-5 (1:500, Thermo Fisher Scientific, catalog number MA5-12808), and β-actin (1:10,000, Cell Signaling Technology, catalog number catalog number 3700). The next day, the blots were washed three times in TBS-T for 10 minutes and incubated in the specific secondary antibodies (1:20,000, LI-COR Biosciences, catalog numbers 926-68020 and 926-32211) for 1 hour at room temperature. The blots were then washed with TBS-T, and imaged/quantified using a LICOR Odyssey CLx (LI-COR Biosciences) attached to a Dell computer (OptiPlex 7010) running Windows 7 and Image Studio (version 1.0.11, LI-COR Biosciences). For the detection of MLKL monomers and dimers, proteins were run in 3-8% tris-acetate gel (Thermo Fisher Scientific) in non-reducing conditions. The ELISA experiments to measure Aβ levels were done following the protocol we detailed in previous publications⁴⁹.

Antibodies validation:

Western blots. To validate the RIPK1 antibody, we loaded on a gel proteins extracted from wild type cells, RIPK1 knockout cells (Horizon Discovery, Cambridge, United Kingdom, Catalog number HZGHC000060c015), RIPK1 knockout cells transfected with a RIPK1 expressing plasmid (Addgene, Cambridge, MA, Catalog number 78842), CTL and AD human cases, non-transgenic and 5xFAD mice. Notably, the expected band of 73 kDa (arrow in FIG. 23a) was not present in the knockout cells. To validate the RIPK3 antibody, we loaded on a gel proteins extracted from wild type mice, RIPK3 knockout mice (obtained from Genentech, South San Francisco, Calif.), wild type cells transfected with a RIPK3 expressing plasmid (Addgene, Cambridge, Mass., catalog number 41387), CTL and AD human cases, non-transgenic and 5xFAD mice. The expected band of 55 kDa (arrow in FIG. 23b) was not present in the RIPK3 knockout mice and was present in the cells transfected with the RIPK3-expressing plasmid. Notably, in this case the band ran a little slower, as the plasmid has a GFP tag to its C-terminal. To validate the MLKL antibody, we loaded on a gel proteins extracted from wild type cells, MLKL knockout cells (Horizon Discovery, Cambridge, United Kingdom, catalog number HXGHC000712c013), MLKL knockout cells transfected with a MLKL-expressing plasmid pRetroX-TRE3G MLKL-Flag (obtained from Dr. Patrick Fitzgerald, St. Jude Children's Research Hospital, Memphis, Tenn.), CTL and AD human cases, and non-transgenic and 5xFAD mice. Notably, the expected band of 51 kDa (arrow in FIG. 23c) was not present in the knockout cells, but it was present when these cells were transfected with an MLKL plasmid. To validate the phospho-specific MLKL antibody, we loaded on a gel protein extracted from MLKL knockout and wild type cells. As a positive control, cells were treated with 1 ng/mL TNFa (PeproTech) and 50 μM pan caspase inhibitor zVAD-FMK (R&D Systems) for 4 hours prior to protein extraction. This is a known protocol to induce necroptosis in vitro²⁶. Proteins extracted from NonTg and 5xFAD mice were also added. As shown in FIG. 23d, we detected a strong band in the positive control but not in the MLKL knockout cells.

Immunohistochemistry. To validate the RIPK1, RIPK3, and MLKL antibodies for immunohistochemistry, we used the knockout out cell lines for RIPK1 and MLKL listed above with their matched control (HAP-1 cell line), and primary fibroblasts isolated from RIPK3 knockout and wild type mice. As shown in FIG. 24a, only background staining was evident in the knockout cells. To validate the pMLKL antibodies, we induced necroptosis in HAP-1 cells with 1 ng/mL TNFα and 50 μM Caspase inhibitor zVAD-FMK for 4 hours. Cells were then stained with the two pMLKL antibodies utilized in this work. A strong immunoreactivity for both pMLKL antibodies was only evident after inducing necroptosis (FIG. 24b).

Immunoprecipitation: Samples from AD and CTL brains were homogenized in TBS/Triton-X100. Protein concentrations were determined using the Bradford assay (Thermo Fischer Scientific). 100 μg of proteins per each sample were incubated with 4 μg of anti-RIPK1 (BD Transduction Laboratories), for 48 hours while rotating at 4° C. After incubation, 100 l of pre-washed Protein G magnetic beads (Biorad) were added to each sample. Samples and beads were incubated at 4° C. for 3 hours. The beads were then washed three times with TBS-T and mixed with 13.5 μl of 4× loading buffer containing 1.5 μl reducing buffer (Thermo Fisher Scientific). The beads were boiled at 90° C. for 5 min and placed on ice. Samples were loaded onto a 10% Bis-Tris gel in MOPS running buffer (Thermo Fisher Scientific). Gels were transferred to a nitrocellulose membrane and incubated overnight at 4° C. with anti-MLKL (1:1000, Novus Biologicals) followed by one hour incubation at room temperature with anti-rabbit IgG-HRP (1:20,000, Thermo Fisher Scientific). Blots were then washed and developed with Super Signal West Dura (Thermo Fisher Scientific) and processed on film. The quantification of the bands was performed using ImageJ.

RNA extraction and microarray analysis: The total RNA extracted from frozen temporal gyms tissue was isolated with RNEasy Mini Kit (Qiagen) starting with at least 60 mg of tissue. Samples were prepared using TargetAMp—Nano labeling Kit (Ilumina) and hybridized to HumanHT-12 v4 BeadChip array (Illumina) according to manufacturer's protocol. The raw data were background corrected using Genome Studio data analysis software (Illumina), and then exported for following analysis. For quality control purposes, hierarchical clustering, MDS, and density plots were inspected to detect outliers and low quality samples using the R package ArrayQualityMetrics⁵⁰. The expression dataset was annotated with the R-package lumiHumanAll.db, originally consisting of 42,179 informative probes. After filtering for low expressed and non-annotated probes we obtained a final dataset of 26,583 probes corresponding to 21,122 unique genes. The HumanHT-12v4 BeadChip array contains multiple probes matching a single gene; however, no summarization of the data was performed in order to avoid the loss of information because different probes can correspond to multiple transcript variants/isoforms for the same gene. Variance of raw data was stabilized using a variance-stabilizing transformation (VST), and the data were normalized using the quantile method by using the lumiExpresso function with default parameters. The moderated t-statistics, as well as FDR correction method, were used for detection of differential expression and P-value correction, respectively⁵¹. Transcripts with adjusted P value<0.05 were considered statistically significant. VST and differential expression analysis were conducted using the R-package Lumi⁵². The normalized expression values for RIPK1 (NM_003804), MLKL (NM_152649) and RIPK3 (NM_006871) from the microarray experiments are available as supplementary material (Supplementary File 1).

For validation purposes, we reanalyzed the publicly available dataset GSE5281. Data were processed with the Affymetrix U133 Plus 2.0 array. In this data set, there were samples obtained from the tissue bank at the Banner Sun Health Research Institute and samples obtained from the tissue bank at Washington University. In order to obtain an independent cohort, we excluded the samples obtained from the Banner Sun Health Research Institute. The raw data (*.CEL files) were analyzed applying the Robust Multi-Array Average (RMA) normalization method ⁵³. Differential expression was computed using a linear model, and the significance was assessed using the moderated-t statistics as implemented in the limma R-package⁵⁴. The p-values were corrected using the FDR correction method.

Construction of RIPK1 causal gene regulatory network: We performed causal inference testing²³ to build a causal gene regulatory network focused on RIPK1 in post-mortem brain tissue. This approach requires paired gene expression and genotype data for a large number of samples to establish the direction of regulation between RIPK1 and its correlated genes. RNA sequencing data was obtained from the Accelerating Medicines Partnership—Alzheimer's disease (AMP-AD) Knowledge Portal (synapse ID: syn3157743). Post-mortem samples were collected from the superior temporal gyms (STG, Brodmann Area 22), anterior prefrontal cortex (APFC, Brodmann Area 10), ectorhinal cortex (ERC, Brodmann Area 36) and inferior frontal gyms (IFG, Brodmann Area 44) by the Mount Sinai NIH Brain and Tissue Repository. Samples with RIN less than 6, were removed from the analysis. Single end reads were aligned reads to human genome reference (GRCh37 ensembl version 70 ⁵⁵), using STAR-RNAseq (2.4.0g1) read aligner⁵⁶, and accepted mapped reads were summarized to gene level using the featureCounts function of the subread software package^{57, 58}. Genes with at least 1 count per million mapped reads⁵⁹ in at least half of the sample libraries were retained, and normalized using the voom function in the Limma package^{54, 60}.

Whole Exome Sequencing data used in this study can be obtained from the Accelerating Medicines Partnership - Alzheimer's Disease (AMP-AD) Knowledge Portal (synapse ID : syn4645334). Reads were aligned to human genome hg19 using BWA aligner⁶¹. DNA sequence variants were called using the DNAseq Variant Analysis workflow of GATK Best Practices version 3⁶². Variants with a minor allele frequency<0.05, or with missing calls in >10 samples were removed from further analysis. Common variants were imputed using IMPUTE2^{63, 64}, using 1000 Genomes Phase 3 reference genotypes⁶⁵.

We used the Matrix eQTL software⁶⁶, to identify cis-eQTLs for RIPK1, classifying DNA markers within 1MB of RIPK1 gene boundaries that significantly associate with RIPK1 expression (controlling for age, sex, ethnicity, RNA-sequencing batch, RIN and PMI as covariates), assuming an additive linear model for associating genotype dosage with RIPK1 expression. We classified DNA markers with an association FDR<0.25 as RIPK1 cis-eQTL, identifying multiple markers in two of the four brain regions, the ERC and APFC.

We then identified the subset of these RIPK1 cis-eQTLs that are conditionally independent in their association with RIPK1, and used these for causal inference testing. For each brain region, we ranked cis-eQTL according to ascending P-value, and recursively attempted cis-eQTL discovery while conditioning on the genotypes for cis-eQTL that were detected in any previous iteration. We continued over this process until no more cis-eQTL were discovered, and nominated these loci as “independent RIPK1 cis-eQTL” for that specific brain region. In total, we identified two independent RIPK1 cis-eQTL in the APFC (rs2295768:2954602:C:T, rs13204531:2956846:A:G) and two in the ERC (rs2064310:3080324:C:A, rs4997140:3254710:A:G).

Causal inference testing was then applied to the RNA-seq and genotype data used to detect RIPK1 cis-eQTL in the APFC and ERC. This comprised data from individuals with a range of CERAD neuropathology classification⁶⁷ scores, which was then used to build RIPK1 networks in the APFC (Total individuals: n=174, Controls: n=63, Definite AD: n=68, Likely AD: n=20, Possible AD: n=23) and also the ERC (Total individuals: n=80, Controls: n=33, Definite AD: n=28, Likely AD: n=8, Possible AD: n=11).

Causal inference testing (CIT) has been well described previously²³. Briefly, it offers a hypothesis test for whether a molecule (in this case, the expression of RIPK1) is potentially mediating a causal association between a DNA locus, and some other quantitative trait (such as the expression of genes correlated with RIPK1). Causal relationships can be inferred from a chain of mathematic conditions, requiring that for a given trio of loci (L), a potential causal mediator i.e., RIPK1 (G) and a quantitative trait (T), the following conditions must be satisfied to establish that G is a causal mediator of the association between L and T:
(a) L and G are associated
(b) L and T are associated
(c) L is associated with G, given T
(d) L is independent of T, given G

Although CIT includes tests for linkage (conditions a and b), to control the number of candidate L/G/T trios that are submitted to the CIT function, we perform multiple pre-filtering steps, which are aimed at establishing association between L and G, and L and T, before we submit a particular trio for CIT. Association between L and G is established in the course of the RIPK1 cis-eQTL analysis (described above). Nominal association between L and T is established using matrix eQTL⁶⁶ retaining candidate T molecules (for that specific cis-eQTL) with an association P-value<0.05.

While CIT outputs a P-value, it is actually the highest P-value of the four constituent hypothesis tests, reflecting each of the conditions required to establish causal mediation. This results in a non-uniform CIT P-value distribution under null conditions, which can make appropriate multiple test correction unreliable. To overcome this, we employed a permutation based approach to assess the significance of candidate causal relationships, where candidate traits (T) are randomly shuffled, separately within each genotype dosage group (0, 1 or 2) for each permutation. The false discovery rate was estimated by counting the proportion of permutations (1000 per trio) with a CIT P-value lower than the test CIT P-value.

To minimize the number of false positive inferences, we performed two separate tests for each candidate trio. We tested models that include RIPK1 expression (G) as causal for transgene expression (T) (“causal model”) and separately, the G being regulated by the T (“reactive model”). We thus classified candidate genes (T) as regulated by RIPK1, if they achieved a “causal model” FDR<0.05 and a “reactive model” FDR>0.05. (See Table 11 for full causal inference testing results).

Gene set enrichment testing: We then separated the downstream neighbors of RIPK1 into directed sets, subdividing according to whether they are positively or negatively regulated by RIPK1 and calculated gene set enrichments using Fisher's exact text, and one-sided P-values (to identify over-representation of gene sets) were adjusted using the Benjamini-Hochberg method⁵¹. Gene sets used throughout the enrichment analysis were derived from the molecular signatures database⁶⁸, as well as multiple expression profiles of AD brains obtained from the literature^{18, 24}.

Genetic disease associations shown in FIG. 5 are based on a combination of data from the Human Gene Mutation Database⁶⁹, Online Mendelian Inheritance in Man⁷⁰, ClinVar⁷¹, Gwas Catalog⁷² and Harmonizome⁷³. The RIPK1 network was visualized using Circlize⁷⁴. Network generation and gene set enrichments were performed using the R project for statistical computing version 3.2.5.

Morris water maze: To assess spatial learning and memory, we perform the Morris water maze test, which was conducted during the light cycle in a circular tank of 1.5 meters in diameter located in a room with extra maze cues. The tank was filled with water maintained at 23° C. throughout the duration of the testing. Non-toxic white paint was added to the water to make it opaque. A 14 cm wide platform was kept 1.5 cm beneath the surface of the water, and thus was not visible to mice during the test. The mice received four trial per day for 5 consecutive days. If a mouse was not able to find the platform within 60 seconds, it was gently placed on the platform by the researcher and kept it there for 10 seconds. At the end of each trial, the mice were placed in a warm cage for 25 seconds before starting the next trial. The probe trials were performed 24 hours after the last training trial. For the probe trials the platform was removed and the mice were allowed to swim for 60 seconds. A video camera placed on the ceiling recorded the training and probe trials for each mouse. The results were analyzed using the EthoVisioXT tracking system. The experimenters were blinded to genotype and treatment.

Viral constructs generation and injections: AAVs were generated by Vector BioLabs (Malvern, Pa.) with the following constructs: AAV1-CamKIIa-caMLKL-2A-eGFP-WPRE and AAV1-CamKIIa-eGFP-WPRE for the experimental and control viruses, respectively. The final titers for each virus was 3.8×10′³ GC/ml. Using a 5 μl Hamilton syringe, 2 μl of viral suspension were injected into the third ventricle of anesthetized mice. Mice were anesthetized with isoflurane. Using bregma as a reference point, the stereotaxic coordinates of the injections were: −2.46 mm antero-posterior; 0 mm lateral; 2 mm dorso-ventral from the skull. The virus was injected at 0.5 μl/minute, after which the needle is left in place for 3 additional minutes before it was slowly removed. The experimenter was blinded to the genotype of the mice.

Primary neuronal cultures—For the GFP experiments: Primary neurons were isolated from APP/PS1 and NonTg Po pups. The tissue was then dissociated in Neurobasal media (Thermo Fisher Scientific) and spun down for 3 minutes at 5,000 g at 4° C. The supernatant was discarded and the pellet was re-suspended in 1 ml of Neurobasal media supplemented with B27 (Thermo Fisher Scientific). An equal volume per each sample was seeded into a Poly-D-Lysine-coated (10 mg/mL, Sigma-Aldrich) black 96-well tissue culture plate. After 24 hours in culture, the neurons were infected with 1μl/ml CamKIIa-GFP AAV (virus titer 1.0×10¹³ GC/ml) and at day 2 post infections the neurons were treated either with 10 μM 7-Cl-O-necrostatin-1S (Nec-1S; Millipore) or DMSO (Sigma-Aldrich). Neurons were treated for a total of 15 days and the GFP fluorescence was measured every 4 days. For every measurement fresh media with or without Nec1S was added to the wells. All fluorescent measurements (excitation 488 nm, emission 509 nm) were performed using the Synergy HT multi-mode microplate reader with the Gen5 software (BioTek, Winooski, Vt.). The reader was pre-heated to 37° C. to maintain optimal conditions for the neurons during the measurements. The experimenter was blinded to group allocation. For the NeuN experiments: Neurons were extracted using the same protocol we used for the GFP experiments, plated on round coverslips of 13 mm in diameter coated with Poly-D-Lysine-coated (10 mg/mL, Sigma Aldrich), and placed into 24-well plates. After 7 days in vitro (DIV), neurons were treated with 10 μM 7-Cl-O-necrostatin-1S (Nec-1S; Millipore) or DMSO (Sigma-Aldrich). After the treatment, the remaining plates were fixed with 4% paraformaldehyde and stored at 4° C. at 11 DIV and at 15 DIV. At the end of the experiment neurons per each time point were stained using a NeuN antibody (1:200, Millipore) and mounted in slides using a mounting media with DAPI (Thermo Fisher Scientific). Four fields per coverslip were imaged using a 40× oil objective. Lasers at 405 nm and 488 nm were respectively used to image DAPI and NeuN. Image fields were selected in an unbiased manner.

Nec1S in vivo treatment: To reduce necroptosis in vivo, we used the same treatment paradigm used by¹³. Briefly, 8-month-old 5xFAD and NonTg mice were given a single intraperitoneal injection of 10 mg/kg Nec-1S or vehicle (Focus Biomolecules). Mice were then given 0.5 mg/ml Nec-1S or vehicle in their drinking water, which contained 2% sucrose, for 21 days.

Statistical Analyses: For WB and IF data, the statistical analyses were conducted using GraphPad Prism 5. Sample description and regression statistics were conducted using the R software. Samples description: The age distribution in AD and CTL cases was assessed with mean, standard deviation, and quartiles. The comparison was conducted using a t-test for equal variance, after assessing the normal distribution with the Shapiro-Wilk test and the variance homoscedasticity with the Levene's test. The distribution of gender and ApoE genotypes in AD cases and controls was assessed with Fisher's Exact test with 20,000 simulated P-values. Variables description: The normality of distribution of continuous variables (Height, Weight, BMI, Brain Weight) and MMSE, considered a continuous interval variable, was assessed with the Shapiro-Wilk test and by visual inspection of the distribution plots. MMSE showed a strong departure from normality, due to the bimodal distribution confirmed by Hartigan Test. The distribution between AD cases and controls was assessed using the t-test with equal variance, after assessing homoscedasticity with the Levene's test (normal distributed variables) and non-parametric Mann-Whitney U test (non-normal distributed variables). The distribution of ordinal variables (Braak stage and plaque density) between AD cases and controls was assessed using the Fisher's Exact test with 20,000 simulated P-values. Regression analysis: For continuous variables (Brain Weight), the linear relationship between gene expression and variables was first assessed using a Pearson's correlation. For variables showing a significant correlation (P<0.05), we conducted a linear regression analysis. For ordinal variables (Braak stage and plaque density), we conducted an ordinal logistic regression analysis. Finally, for MMSE we conducted a quantile regression analysis due to the bimodal distribution, running 10 models from 10th to the 100th percentiles and correcting p-values with the FDR method⁵¹. For western blot and IHC, a two-tailed unpaired t test was used to analyze select pairwise comparisons as specified in the results section. Pearson correlation coefficients corresponding to co-localization in double labeled IHC samples, Costes randomized p-values were obtained using ImageJ (NIH). Where representative images are shown, statistical analyses were done on all the samples. A priori power analyses were not performed but our sample sizes are similar to those reported in previously published papers.

References

1. Alzheimer's, A. 2015 Alzheimer's disease facts and figures. Alzheimer's & dementia: the journal of the Alzheimer's Association 11, 332-384 (2015).

2. Liu-Seifert, H., et al. Cognitive and functional decline and their relationship in patients with mild Alzheimer's dementia. J Alzheimers Dis 43, 949-955 (2015).

3. Zahodne, L. B., Manly, J. J., MacKay-Brandt, A. & Stern, Y. Cognitive declines precede and predict functional declines in aging and Alzheimer's disease. PloS one 8, e73645 (2013).

4. Querfurth, H. W. & LaFerla, F. M. Alzheimer's disease. N Engl J Med 362, 329-344 (2010).

5. Khan, S. S. & Bloom, G. S. Tau: The Center of a Signaling Nexus in Alzheimer's Disease. Front Neurosci 10, 31 (2016).

6. Serrano-Pozo, A., Frosch, M. P., Masliah, E. & Hyman, B. T. Neuropathological alterations in Alzheimer disease. Cold Spring Harbor perspectives in medicine 1, a006189 (2011).

7. Caselli, R.J., Beach, T. G., Yaari, R. & Reiman, E. M. Alzheimer's disease a century later. J Clin Psychiatry 67, 1784-1800 (2006).

8. Lalaoui, N., Lindqvist, L. M., Sandow, J. J. & Ekert, P. G. The molecular relationships between apoptosis, autophagy and necroptosis. Seminars in cell & developmental biology 39, 63-69 (2015).

9. Hanson, B. Necroptosis: A New Way of Dying? Cancer Biol Ther, 0 (2016).

10. Linkermann, A. & Green, D. R. Necroptosis. N Engl J Med 370, 455-465 (2014).

11. Cho, Y. S., et al. Phosphorylation-driven assembly of the RIP1-RIP3 complex regulates programmed necrosis and virus-induced inflammation. Cell 137, 1112-1123 (2009).

12. Vandenabeele, P., Galluzzi, L., Vanden Berghe, T. & Kroemer, G. Molecular mechanisms of necroptosis: an ordered cellular explosion. Nat Rev Mol Cell Biol 11, 700-714 (2010).

13. Ofengeim, D., et al. Activation of necroptosis in multiple sclerosis. Cell reports 10, 1836-1849 (2015).

14. Re, D. B., et al. Necroptosis drives motor neuron death in models of both sporadic and familial ALS. Neuron 81, 1001-1008 (2014).

15. Ito, Y., et al. RIPK1 mediates axonal degeneration by promoting inflammation and necroptosis in ALS. Science (New York, N.Y. 353, 603-608 (2016).

16. Li, J., et al. The RIP1/RIP3 necrosome forms a functional amyloid signaling complex required for programmed necrosis. Cell 150, 339-350 (2012).

17. Costes, S. V., et al. Automatic and quantitative measurement of protein-protein colocalization in live cells. Biophys J 86, 3993-4003 (2004).

18. Liang, W. S., et al. Gene expression profiles in anatomically and functionally distinct regions of the normal aged human brain. Physiol Genomics 28, 311-322 (2007).

19. Liang, W. S., et al. Alzheimer's disease is associated with reduced expression of energy metabolism genes in posterior cingulate neurons. Proceedings of the National Academy of Sciences of the United States of America 105, 4441-4446 (2008).

20. Braak, H. & Braak, E. Staging of Alzheimer's disease-related neurofibrillary changes. Neurobiology of aging 16, 271-278; discussion 278-284 (1995).

21. Giacobini, E. & Gold, G. Alzheimer disease therapy—moving from amyloid-beta to tau. Nature reviews. Neurology 9, 677-686 (2013).

22. Arevalo-Rodriguez, I., et al. Mini-Mental State Examination (MMSE) for the detection of Alzheimer's disease and other dementias in people with mild cognitive impairment (MCI). Cochrane Database Syst Rev, CD010783 (2015).

23. Millstein, J., Zhang, B., Zhu, J. & Schadt, E. E. Disentangling molecular relationships with a causal inference test. BMC Genet 10, 23 (2009).

24. Blalock, E. M., et al. Incipient Alzheimer's disease: microarray correlation analyses reveal major transcriptional and tumor suppressor responses. Proceedings of the National Academy of Sciences of the United States of America 101, 2173-2178 (2004).

25. Hitomi, J., et al. Identification of a molecular signaling network that regulates a cellular necrotic cell death pathway. Cell 135, 1311-1323 (2008).

26. Liu, Q., et al. Akt and mTOR mediate programmed necrosis in neurons. Cell death & disease 5, e1084 (2014).

27. Oakley, H., et al. Intraneuronal beta-amyloid aggregates, neurodegeneration, and neuron loss in transgenic mice with five familial Alzheimer's disease mutations: potential factors in amyloid plaque formation. J Neurosci 26, 10129-10140 (2006).

28. Jankowsky, J. L., et al. Mutant presenilins specifically elevate the levels of the 42 residue beta-amyloid peptide in vivo: evidence for augmentation of a 42-specific gamma secretase. Human molecular genetics 13, 159-170 (2004).

29. Rodriguez, D. A., et al. Characterization of RIPK3-mediated phosphorylation of the activation loop of MLKL during necroptosis. Cell death and differentiation 23, 76-88 (2016).

30. Dickerson, B. C., et al. Alzheimer-signature MM biomarker predicts AD dementia in cognitively normal adults. Neurology 76, 1395-1402 (2011).

31. Popescu, B. O. & Ankarcrona, M. Mechanisms of cell death in Alzheimer's disease: role of presenilins. J Alzheimers Dis 6, 123-128 (2004).

32. Bancher, C., Lassmann, H., Breitschopf, H. & Jellinger, K. A. Mechanisms of cell death in Alzheimer's disease. J Neural Transm Suppl 50, 141-152 (1997).

33. Zhu, X., Raina, A. K., Perry, G. & Smith, M. A. Apoptosis in Alzheimer disease: a mathematical improbability. Current Alzheimer research 3, 393-396 (2006).

34. Niikura, T., Tajima, H. & Kita, Y. Neuronal cell death in Alzheimer's disease and a neuroprotective factor, humanin. Curr Neuropharmacol 4, 139-147 (2006).

35. Cotman, C. W. & Su, J. H. Mechanisms of neuronal death in Alzheimer's disease. Brain pathology (Zurich, Switzerland) 6, 493-506 (1996).

36. Abraham, M. C. & Shaham, S. Death without caspases, caspases without death. Trends in cell biology 14, 184-193 (2004).

37. Snigdha, S., Smith, E. D., Prieto, G. A. & Cotman, C. W. Caspase-3 activation as a bifurcation point between plasticity and cell death. Neurosci Bull 28, 14-24 (2012).

38. Stadelmann, C., Bruck, W., Bancher, C., Jellinger, K. & Lassmann, H. Alzheimer disease: DNA fragmentation indicates increased neuronal vulnerability, but not apoptosis. J Neuropathol Exp Neurol 57, 456-464 (1998).

39. Perry, G., Nunomura, A., Lucassen, P., Lassmann, H. & Smith, M. A. Apoptosis and Alzheimer's disease. Science (New York, N.Y. 282, 1268-1269 (1998).

40. Vanden Berghe, T., Kaiser, W. J., Bertrand, M. J. & Vandenabeele, P. Molecular crosstalk between apoptosis, necroptosis, and survival signaling. Mol Cell Oncol 2, e975093 (2015).

41. Zheng, C., Zhou, X. W. & Wang, J. Z. The dual roles of cytokines in Alzheimer's disease: update on interleukins, TNF-alpha, TGF-beta and IFN-gamma. Transl Neurodegener 5, 7 (2016).

42. Dillon, C. P., et al. RIPK1 blocks early postnatal lethality mediated by caspase-8 and RIPK3. Cell 157, 1189-1202 (2014).

43. Kaiser, W. J., et al. RIP1 suppresses innate immune necrotic as well as apoptotic cell death during mammalian parturition. Proceedings of the National Academy of Sciences of the United States of America 111, 7753-7758 (2014).

44. Dondelinger, Y., et al. MLKL compromises plasma membrane integrity by binding to phosphatidylinositol phosphates. Cell reports 7, 971-981 (2014).

45. Su, L., et al. A plug release mechanism for membrane permeation by MLKL. Structure 22, 1489-1500 (2014).

46. Wang, H., et al. Mixed lineage kinase domain-like protein MLKL causes necrotic membrane disruption upon phosphorylation by RIP3. Molecular cell 54, 133-146 (2014).

47. Nelson, P. T., et al. Correlation of Alzheimer disease neuropathologic changes with cognitive status: a review of the literature. J Neuropathol Exp Neurol 71, 362-381 (2012).

48. Beach, T. G., et al. The Sun Health Research Institute Brain Donation Program: description and experience, 1987-2007. Cell and tissue banking 9, 229-245 (2008).

49. Caccamo, A., Ferreira, E., Branca, C. & Oddo, S. p62 improves AD-like pathology by increasing autophagy. Molecular psychiatry (2016).

50. Kauffmann, A., Gentleman, R. & Huber, W. arrayQualityMetrics--a bioconductor package for quality assessment of microarray data. Bioinformatics 25, 415-416 (2009).

51. Benjamini, Y. & Hochberg, Y. Controlling the False Discovery Rate: A Practical and Powerful Approach to Multiple Testing. Journal of the Royal Statistical Society. Series B (Methodological) 57, 289-300 (1995).

52. Du, P., Kibbe, W. A. & Lin, S. M. Iumi: a pipeline for processing Illumina microarray. Bioinformatics 24, 1547-1548 (2008).

53. Irizarry, R. A., et al. Exploration, normalization, and summaries of high density oligonucleotide array probe level data. Biostatistics 4, 249-264 (2003).

54. Ritchie, M.E., et al. limma powers differential expression analyses for RNA-sequencing and microarray studies. Nucleic Acids Res 43, e47 (2015).

55. Kersey, P. J., et al. Ensembl Genomes 2016: more genomes, more complexity. Nucleic Acids Res 44, D574-580 (2016).

56. Dobin, A., et al. STAR: ultrafast universal RNA-seq aligner. Bioinformatics 29, 15-21 (2013).

57. Liao, Y., Smyth, G. K. & Shi, W. The Subread aligner: fast, accurate and scalable read mapping by seed-and-vote. Nucleic Acids Res 41, e108 (2013).

58. Liao, Y., Smyth, G. K. & Shi, W. featureCounts: an efficient general purpose program for assigning sequence reads to genomic features. Bioinformatics 30, 923-930 (2014).

59. Robinson, M. D., McCarthy, D. J. & Smyth, G. K. edgeR: a Bioconductor package for differential expression analysis of digital gene expression data. Bioinformatics 26, 139-140 (2010).

60. Law, C. W., Chen, Y., Shi, W. & Smyth, G. K. voom: Precision weights unlock linear model analysis tools for RNA-seq read counts. Genome Biol 15, R29 (2014).

61. Li, H. & Durbin, R. Fast and accurate short read alignment with Burrows-Wheeler transform. Bioinformatics 25, 1754-1760 (2009).

62. Van der Auwera, G. A., et al. From FastQ data to high confidence variant calls: the Genome Analysis Toolkit best practices pipeline. Curr Protoc Bioinformatics 43, 11 10 11-33 (2013).

63. Howie, B., Marchini, J. & Stephens, M. Genotype imputation with thousands of genomes. G3 (Bethesda) 1, 457-470 (2011).

64. Howie, B. N., Donnelly, P. & Marchini, J. A flexible and accurate genotype imputation method for the next generation of genome-wide association studies. PLoS Genet 5, e1000529 (2009).

65. Genomes Project, C., et al. A global reference for human genetic variation. Nature 526, 68-74 (2015).

66. Shabalin, A. A. Matrix eQTL: ultra fast eQTL analysis via large matrix operations. Bioinformatics 28, 1353-1358 (2012).

67. Mirra, S. S., et al. The Consortium to Establish a Registry for Alzheimer's Disease (CERAD). Part II. Standardization of the neuropathologic assessment of Alzheimer's disease. Neurology 41, 479-486 (1991).

68. Subramanian, A., et al. Gene set enrichment analysis: a knowledge-based approach for interpreting genome-wide expression profiles. Proceedings of the National Academy of Sciences of the United States of America 102, 15545-15550 (2005).

69. Stenson, P. D., et al. The Human Gene Mutation Database: 2008 update. Genome Med 1, 13 (2009).

70. Hamosh, A., Scott, A. F., Amberger, J., Valle, D. & McKusick, V. A. Online Mendelian Inheritance in Man (OMIM). Hum Mutat 15, 57-61 (2000).

71. Landrum, M. J., et al. ClinVar: public archive of interpretations of clinically relevant variants. Nucleic Acids Res 44, D862-868 (2016).

72. Welter, D., et al. The NHGRI GWAS Catalog, a curated resource of SNP-trait associations. Nucleic Acids Res 42, D1001-1006 (2014).

73. Rouillard, A. D., et al. The harmonizome: a collection of processed datasets gathered to serve and mine knowledge about genes and proteins. Database (Oxford) 2016 (2016).

74. Gu, Z., Gu, L., Eils, R., Schlesner, M. & Brors, B. circlize Implements and enhances circular visualization in R. Bioinformatics 30, 2811-2812 (2014).

All references listed in this application are incorporated by reference for all purposes. While specific embodiments and examples of the disclosed subject matter have been discussed herein, these examples are illustrative and not restrictive. Many variations will become apparent to those skilled in the art upon review of this specification and the claims below.

We claim:

1. A method of reducing neuronal loss in a subject having or suspected of having a neurodegenerative disease, the method comprising the step of administering to the subject a therapeutically effective amount of a compound that inhibits a necroptosis-associated activity of receptor-interactive protein kinase 1 (RIPK1), RIPK3, or Mixed Lineage Kinase Domain-like (MLKL), or a pharmaceutically acceptable salt thereof, whereby neuronal loss is reduced in the subject.

2. The method of claim 1, wherein the compound is a small molecule inhibitor of RIPK1, RIPK3, or MLKL.

3. The method of claim 2, wherein the small molecule inhibitor is 7-Cl-O-necrostatin-1S (Nec-1S), necrosulfonamide, or GSK'872, or a pharmaceutically acceptable salt thereof

4. The method of claim 1, wherein the compound inhibits phosphorylation of MLKL at an amino acid position selected from the group consisting of position 357 and position 358, wherein the position is numbered relative to SEQ ID NO:2.

5. The method of claim 1, wherein the compound inhibits formation of MLKL homodimers.

6. The method of claim 1, wherein the neurodegenerative disease is a neurodegenerative disease of the central or peripheral nervous system.

7. The method of claim 1, wherein the neurodegenerative disease is selected from the group consisting of Alzheimer's disease (AD), multiple sclerosis (MS), Huntington Disease (HD), amyotrophic lateral sclerosis (ALS), and Down Syndrome.

8. A method of treating neuron loss in a subject having or suspected or having a condition associated with aberrant necroptosis activation, the method comprising administering to the subject a therapeutically effective amount of a compound that inhibits a necroptosis-associated activity of RIPK1, RIPK3, or MLKL, or a pharmaceutically acceptable salt thereof, whereby neuronal loss is reduced in the subject.

9. The method of claim 8, wherein the compound is a small molecule inhibitor of RIPK1, RIPK3, or MLKL.

10. The method of claim 9, wherein the small molecule inhibitor is 7-Cl-O-necrostatin-1S (Nec-1S), necrosulfonamide, or GSK'872, or a pharmaceutically acceptable salt thereof

11. The method of claim 8, wherein the compound inhibits phosphorylation of MLKL at an amino acid position selected from the group consisting of position 357 and position 358, wherein the position is numbered relative to SEQ ID NO:2.

12. The method of claim 8, wherein the compound inhibits formation of MLKL homodimers.

13. The method of claim 8, wherein the condition associated with aberrant necroptosis activation is a neurodegenerative disease of the central or peripheral nervous system.

14. The method of claim 13, wherein the neurodegenerative disease is selected from the group consisting of Alzheimer's disease (AD), multiple sclerosis (MS), Huntington's Disease (HD), Parkinson's Disease (PD), amyotrophic lateral sclerosis (ALS), and Down Syndrome.


SEQUENCE LISTING (2 sequences)

SEQ ID NO: 1 - DNA, 2779 bp, Homo sapiens
aatattatccagcacagagaagcaaacaccagaagcttctctccaactttcagtttttctctaaacttccagtccttctcatactgtgtcctggttataatagttgctgggaccttattctattctgaatccccagcccctaaagaaggcttggtactgacgtgtacagcgtagttacccagtgactttggggaaggcaggaagagtgtggaaaggacaggaggaaggaagggaggggccgtggctccgagctgcctagaaagcggctccagggagccagggcaccgtgagcctggtggttggcagctggagccacgtcggagggggaagtgtcgcagcattctctgcaggcatcacagacctgaggcagtggcctccggagggcactggacagaaacagccatccaagtggctgagtggagggaccctgctcaagtgcagctgcagtggccggggtttccctcaggtagggatcggggcgccttgtcgccgccagccacgtgtggcgtccggtacagtcagcagagtgcagggtgcgggcaccaggaaagggggcgcaggggaactcccgcgggcctcgcgtttgcaaacttctcgcctgggcaggaggcggtcgtgggaaagaaggtggaagagcgagctttttggaactgtgcacgggacagattggacgcacacccctcgggaggcgcgaaggcatggaaaatttgaagcatattatcacccttggccaggtcatccacaaacggtgtgaagagatgaaatactgcaagaaacagtgccggcgcctgggccaccgcgtcctcggcctgatcaagcctctggagatgctccaggaccaaggaaagaggagcgtgccctctgagaagttaaccacagccatgaaccgcttcaaggctgccctggaggaggctaatggggagatagaaaagttcagcaatagatccaatatctgcaggtttctaacagcaagccaggacaaaatactcttcaaggacgtgaacaggaagctgagtgatgtctggaaggagctctcgctgttacttcaggttgagcaacgcatgcctgtttcacccataagccaaggagcgtcctgggcacaggaagatcagcaggatgcagacgaagacaggcgagctttccagatgctaagaagagataatgaaaaaatagaagcttcactgagacgattagaaatcaacatgaaagaaatcaaggaaactttgaggcagtatttaccaccaaaatgcatgcaggagatcccgcaagagcaaatcaaggagatcaagaaggagcagctttcaggatccccgtggattctgctaagggaaaatgaagtcagcacactttataaaggagaataccacagagctccagtggccataaaagtattcaaaaaactccaggctggcagcattgcaatagtgaggcagactttcaataaggagatcaaaaccatgaagaaattcgaatctcccaacatcctgcgtatatttgggatttgcattgatgaaacagtgactccgcctcaattctccattgtcatggagtactgtgaactcgggaccctgagggagctgttggatagggaaaaagacctcacacttggcaagcgcatggtcctagtcctgggggcagcccgaggcctataccggctacaccattcagaagcacctgaactccacggaaaaatcagaagctcaaacttcctggtaactcaaggctaccaagtgaagcttgcaggatttgagttgaggaaaacacagacttccatgagtttgggaactacgagagaaaagacagacagagtcaaatctacagcatatctctcacctcaggaactggaagatgtattttatcaatatgatgtaaagtctgaaatatacagctttggaatcgtcctctgggaaatcgccactggagatatcccgtttcaaggctgtaattctgagaagatccgcaagctggtggctgtgaagcggcagcaggagccactgggtgaagactgcccttcagagctgcgggagatcattgatgagtgccgggcccatgatccctctgtgcggccctctgtggatgaaatcttaaagaaactctccaccttttctaagtagtgtatcaaaatctaaaccaaggagtctctggacaagaagctgggagaggcacaaactggacatctctctctctcatatccttcggcattgggttatctatgggtgcaaggagtgggcacgcttctctgttacaaatagaaaacgattccagtcatacaggacacatcccactccaaatgatatttccaaaaacatacctctgacagtaactttgatagatggtttgtcaaatgtatctttctgggtatccacacctcttggcaatgaaatttgcagctcctcccttccataaatgaagtctctttccccaccatttgaatctgggctggcactgtgacttgatttgatcaatagaatgtggaagaagtgactgtatgccagttccaagcctaggtttcaagaggccttataaatgtctgttggaaccttacccagccatgaacatgttgagtgagcatgctggagaatgagagaccacatgaagcagaaacatgctttcctagctgaagtcatactagcccaaccaacatggcagctaacacatgaatgaggccaatcaagaccagaagaaccactcaagcagatcccagcccaaattgcccattcacacaatcaggagctaaataaattactgttgtcttaacactaaaaaaaaaaaaaaaaaa

SEQ ID NO: 2 - protein, 471 aa, Homo sapiens
MENLKHIITLGQVIHKRCEEMKYCKKQCRRLGHRVLGLIKPLEMLQDQGKRSVPSEKLTTAMNRFKAALEEANGEIEKFSNRSNICRFLTASQDKILFKDVNRKLSDVWKELSLLLQVEQRMPVSPISQGASWAQEDQQDADEDRRAFQMLRRDNEKIEASLRRLEINMKEIKETLRQYLPPKCMQEIPQEQIKEIKKEQLSGSPWILLRENEVSTLYKGEYHRAPVAIKVFKKLQAGSIAIVRQTFNKEIKTMKKFESPNILRIFGICIDETVTPPQFSIVMEYCELGTLRELLDREKDLTLGKRMVLVLGAARGLYRLHHSEAPELHGKIRSSNFLVTQGYQVKLAGFELRKTQTSMSLGTTREKTDRVKSTAYLSPQELEDVFYQYDVKSEIYSFGIVLWEIATGDIPFQGCNSEKIRKLVAVKRQQEPLGEDCPSELREIIDECRAHDPSVRPSVDEILKKLSTFSK